(19) United States
(12) Patent Application Publication
(10) Pub. No.: US 2024/0108271 A1
(43) Pub. Date: Apr. 4, 2024

(54) METHODS AND SYSTEMS FOR DETECTING PACE PULSES

(71) Applicant: Welch Allyn, Inc., Skaneateles Falls, NY (US)

(72) Inventors: Patrick J. Noffke, Hartland, WI (US); Keith Becker, Colgate, WI (US)

(73) Assignee: Welch Allyn, Inc., Skaneateles Falls, NY (US)

(21) Appl. No.: 18/374,662

(22) Filed: Sep. 28, 2023

Related U.S. Application Data
(60) Provisional application No. 63/412,312, filed on Sep. 30, 2022.

(51) Int. Cl. A61B 5/349; G16H 50/20

(52) U.S. Cl. CPC A61B 5/349; G16H 50/20

(57) ABSTRACT

This disclosure relates to methods and systems for detecting pace pulses in electrocardiogram (ECG) data. First ECG data associated with a first sampling frequency can be obtained. Candidate pace pulses in the first ECG data can be identified. Additional data comprising waveform descriptors characterizing the candidate pace pulses can be generated. Second ECG data can be generated by down-sampling the first ECG data. The second ECG data can be associated with a second sampling frequency that is lower than the first sampling frequency. Multiple ECG waveforms can be presented based on the second ECG data. The waveform descriptors characterizing candidate pace pulses can be presented based on the additional data.

### CROSS-REFERENCE TO RELATED APPLICATION

This application claims priority to U.S. Provisional App. No. 63/412,312, which was filed on Sep. 30, 2022 and is incorporated by reference herein in its entirety.

### TECHNICAL FIELD

This application relates to medical devices, and in particular, to methods and systems for detecting pace pulses in electrocardiogram (ECG) data.

### BACKGROUND

An example medical device obtains an ECG by detecting the electrical activity of the heart using electrodes placed in a configuration across a patient's body. The electrodes are electrically coupled to the medical device, which detects differences in voltage over time across different combinations of the electrodes. These relative voltages over time are referred to as “leads.” In various examples, the medical device outputs one or more of the leads to a user. For instance, the medical device may include a display that visually outputs waveforms representing the leads.

An implantable cardiac pacemaker is a medical device configured to treat various heat conditions such as bradycardia and so on. For instance, when the heart rate of a patient is too slow, the pacemaker delivers electrical impulses to the heart of the patient to increase the patient's heart rate. In various cases, implantable cardiac pacemakers deliver bipolar pulses and apply complex algorithms for sensing atrial and ventricular depolarization and for implementing various timing patterns. Some pacemakers utilize biventricular pacing for cardiac resynchronization therapy (CRT).

The electrical impulses generated by a pacemaker impact the ECG detected from a patient in which the pacemaker is implanted. In some cases, an ECG (e.g., a 12-lead ECG or Holter recording) can be used to determine whether a pacemaker is present and assess the performance of the pacemaker. However, the complexity of pacemaker technology can lead to bizarre pacing rhythms on the ECG. In addition, pace pulses may present as fast spikes in the ECG data and are often indistinguishable from electrostatic interference. Moreover, high-frequency artifacts caused by non-cardiac muscle activity can mask pace pulses. Additionally, interference caused by static electricity or other devices can emulate pacemaker activity, leading to false-positive detection of pace pulses. Further, ECG data are usually down-sampled after acquisition and before they are displayed or printed to decrease the temporal resolution of the ECG waveforms. Down-sampling can decrease temporal resolution by reducing the rate of data processed by the display device, which may be associated with limited resources (e.g., storage space and/or bandwidth). In some cases, the ECG data is down-sampled using a lowpass filter, which can filter out high-frequency noise from the ECG data. However, the lowpass filter may also filter out the high-frequency pacemaker activity, such that pace pulses are not apparent in the displayed or printed ECG waveform. Therefore, presenting pace pulses using conventional ECG acquisition devices may present challenges.

### SUMMARY

Various systems and methods disclosed and contemplated herein are directed toward detecting pace pulses in ECG data. In some examples, a frontend device can obtain ECG data via multiple sensors. In some instances, the frontend device can sample an analog ECG signal to obtain high-fidelity first ECG data, in order to detect pacemaker activity, which may include pace pulses as fast as 0.1 milliseconds (msec). The high-fidelity first ECG data can be associated with a first sampling frequency. In some examples, the first sampling frequency can be between 8 kHz and 40 kHz (e.g., including the edges of the range). The frontend device can identify the candidate pace pulses in the first ECG data. In some examples, the frontend device can identify the candidate pace pulses in the first ECG data by identifying, in the first ECG data, a depolarizing pulse. A time window around the detection time for the candidate pace pulse may be used to bound the capture of additional pace related data. The candidate pace pulses may refer to signals in the ECG data that have a similar shape to the true pace pulse. In some examples described herein, the ECG data may or may not include pace pulses. Moreover, there may be interference that mimics pace pulses. Therefore, the frontend device detects signals in the ECG data that have a similar shape to the true pace pulse as “candidate pace pulses.”

The frontend device can generate additional data including waveform descriptors characterizing the candidate pace pulses. In some examples, the waveform descriptors can characterize shapes of the candidate pace pulses. In some examples, the waveform descriptors can characterize frequency components of the candidate pace pulses. In some examples, the waveform descriptors can characterize amplitudes of the candidate pace pulses. For example, the waveform descriptors can include the raw 40 kHz samples for a brief interval, compressed samples, or a representation such as piecewise splines that can accurately represent the pace pulse signal. The additional pace related data can be provided in conjunction with the reduced fidelity data, sufficient for clinical use of the ECG, typically at a second sampling frequency of between 500 Hz and 1000 Hz.

In some examples, generating the additional data can include identifying a portion of the first ECG data including a candidate pace pulse among the candidate pace pulses, and generating a waveform descriptor among the waveform descriptors based on the portion of the first ECG data. In some examples, generating the waveform descriptor among the waveform descriptors can include down-sampling the portion of the first ECG data at a third sampling frequency. The third sampling frequency can be higher than the second sampling frequency and lower than the first sampling frequency. In some examples, the waveform descriptor can include the portion of the first ECG data. The waveform descriptor can include a representation of the portion of the first ECG data. In some examples, the representation can have a lower fidelity than the portion of the first ECG data. In some examples, the waveform descriptor includes one or more frequency components of the portion of the first ECG data.

The frontend device can generate second ECG data by down-sampling the first ECG data. The second ECG data can be associated with a second sampling frequency that is lower than the first sampling frequency. For example, the second sampling frequency is between 500 Hz and 1000 Hz (including the edges of the range). In some examples, the frontend device can pass the first ECG data through a filter. As described herein, the candidate pace pulses can be undetectable if the high-fidelity ECG data are down-sampled without preserving the candidate pace pulses. The techniques described herein can detect and identify the high-fidelity first ECG data with candidate pace pulses at a relatively high frequency (e.g., the first sampling frequency), preserve the candidate pace pulses by generating additional data including waveform descriptors, and then down-sample the high-fidelity first ECG data to generate the second ECG data to lower the volume of the data to save the resources.

The frontend device can transmit the second ECG data and the additional data to a backend device. The backend device can present multiple ECG waveforms respectively corresponding to multiple leads based on the second ECG data. The backend device can present the ECG waveforms and the candidate pace pulses together or separately. For example, the backend device can present multiple ECG waveforms respectively corresponding to multiple leads, and present the waveform descriptors in a user interface element that is separate from the multiple ECG waveforms. Additionally or alternatively, the backend device can present multiple ECG waveforms respectively corresponding to multiple leads, and present the waveform descriptors in a user interface element that is visually overlapping the multiple ECG waveforms.

The backend device can determine that a waveform descriptor among the waveform descriptors includes an artifact. In some examples, the backend device can identify a spike in the waveform descriptor, and determine that a paced rhythm is absent within a threshold time period after the spike in the waveform descriptor. A spike can be a sharp increase in the magnitude in a waveform. A paced rhythm can be an electrocardiographic finding in which the cardiac rhythm is controlled by an electrical impulse from an artificial cardiac pacemaker. In some examples, the backend device can modify the additional data by removing the waveform descriptor comprising the artifact. In some examples, the backend device can perform one or more algorithms on the waveform descriptors. For example, the backend device can perform signal-averaging on candidate pace pulses to generate an average pace pulse. Moreover, the backend device can compare an individual candidate pace pulse with the average pace pulse. Additionally, the backend device can automatically perform and/or provide the ability for the user to compare the average pace pulse to at least one of the individual pace pulse in the waveform descriptors. As another example, the backend device can apply an algorithm to group the individual candidate pace pulses by morphology, and perform signal-averaging on the candidate pace pulses having the same or similar morphology. Morphology can refer to the form of the candidate pace pulse, such as the size, shape, structure, or the like of the candidate pace pulse. Then, a list including different morphologies with representative signal-averaged pace pulses can be generated and presented to the user for user's review and analysis. In some examples, the backend device can determine a discrepancy between the average pace pulse and an individual pace pulse in the waveform descriptors, and determine that the discrepancy is greater than a threshold. The backend device can modify the additional data by removing the individual candidate pace pulse among the waveform descriptors based on determining that the discrepancy is greater than the threshold.

The backend device can include a display. The backend device can receive an input signal indicating a zoom-in instruction. In some examples, the backend device can present, on the display, a zoomed-in view of the at least one ECG waveform based on receiving the input signal. In some examples, the backend device can present, on the display, a zoomed-in view of at least one of the waveform descriptors based on receiving the input signal. In some examples, the backend device can print at least one ECG waveform on a substrate, and print indications of the waveform descriptors on the substrate.

Another aspect of this disclosure provides a system for detecting pace pulses in ECG data. The system can obtain first ECG data associated with a first sampling frequency. The system can identify candidate pace pulses in the first ECG data. The system can generate additional data including waveform descriptors characterizing the candidate pace pulses. The system can generate second ECG data by down-sampling the first ECG data. The second ECG data can be associated with a second sampling frequency that is lower than the first sampling frequency. The system can include a display configured to present multiple ECG waveforms based on the second ECG data, and present the waveform descriptors characterizing the one or more candidate pace pulses based on the additional data.

Techniques discussed herein can provide improvements to methods and systems for detecting candidate pace pulses in ECG data. For example, high-fidelity first ECG data can be obtained, and candidate pace pulses can be identified in the high-fidelity first ECG data. Various types of waveform descriptors can be generated to characterize the candidate pace pulses in terms of frequency, amplitude, shape, or the like. The high-fidelity first ECG data can be down-sampled to generate second ECG data with a lower fidelity to save computing resources (e.g., storage, bandwidth, or the like). Meanwhile, the candidate pace pulse can be preserved in the additional data that includes the waveform descriptors. Moreover, artifacts can be identified and optionally removed from the candidate pace pulses to render more accurate display of pacemaker activity. Moreover, the system can allow a user (such as a cardiologist, a physician, or the like) to quickly review the ECG waveforms with pacemaker activity on a display via a user interface. The user can zoom in on a portion of the ECG waveform or a portion of the waveform descriptor conveniently. Additional clinical insights regarding the pacemaker activity may be gleaned.

### BRIEF DESCRIPTION OF THE DRAWINGS

The following figures, which form a part of this disclosure, are illustrative of the described technology and are not meant to limit the scope of the claims in any manner.

FIG. 1 shows a schematic block diagram of an example ECG system environment in accordance with implementations of this disclosure.

FIG. 2A illustrates example ECG waveforms in accordance with implementations of this disclosure.

FIG. 2B illustrates an example scenario where a portion of an ECG waveform is zoomed in in accordance with implementations of this disclosure.

FIG. 2C illustrates an example view of waveform descriptors characterizing one or more candidate pace pulses in accordance with implementations of this disclosure.

FIG. 2D illustrates an example scenario where a portion of the waveform descriptors is zoomed in in accordance with implementations of this disclosure.

FIGS. 3A, 3B, 3C, 3D, 3E, and 3F illustrate example pace pulses in accordance with implementations of this disclosure.

FIG. 4 illustrates a flow chart of an example process for detecting pace pules in the ECG data in accordance with implementations of this disclosure.

FIG. 5 illustrates at least one example device configured to enable and/or perform the some or all of the functionality discussed herein.

FIG. 6 illustrates a flow chart of an example process for detecting pace pules in the ECG data in accordance with implementations of this disclosure.

FIG. 7 illustrates a flow chart of an example process for detecting pace pules in the ECG data in accordance with implementations of this disclosure.

FIG. 8 illustrates at least one example device configured to enable and/or perform the some or all of the functionality discussed herein.

### DETAILED DESCRIPTION

In various implementations, a frontend device detects an analog ECG of a patient and converts the analog ECG to ECG data, which may include digital data. A backend device analyzes the ECG data and outputs a representation of the ECG data to a user. For example, a clinician may analyze a printout of the ECG data in order to assess the condition of the patient. The backend device may analyze the ECG data for various characteristics, such as identifying the presence of QRS complexes or arrhythmias in the ECG data.

In some cases, the patient has an implantable pacemaker that generates pace pulses during ECG acquisition. The pace pulses are electrical signals that generate spikes in the analog ECG. However, the high frequency of the pace pulses and the fidelity and/or sampling rate of the ECG data may make it difficult, or even impossible, for the backend device to identify the pace pulses in the ECG data. In addition, it can be unfeasible for the backend device to analyze the ECG at a high enough sampling rate to identify the pace pulses, due to latency requirements and/or computing resource constraints at the backend device.

In various implementations of the present disclosure, the frontend device converts the analog ECG to first ECG data having a relatively high sampling frequency and identifies candidate pace pulses in the first ECG data. Further, the frontend device may generate an additional channel that indicates the identified candidate pace pulses. For instance, the additional channel includes waveform descriptors of each of the candidate pace pulses, such as segments of the first ECG data including the identified candidate pace pulses, compressed segments, or other ways of describing characteristics of the candidate pace pulses. In various cases, the frontend device generates second ECG data by down-sampling the first ECG data. The frontend device outputs both the additional channel and the second ECG data to the backend device. In some examples, the frontend device refrains from outputting the first ECG data to the backend device. The backend device, in some instances, outputs the second ECG data and the additional channel to a user. Accordingly, the backend device provides a user with valuable context about potential pacemaker activity in conjunction with the second ECG data. In some cases, the backend device further analyzes the second ECG data in view of the additional channel. In particular examples, the backend device distinguishes between true pace pulses and artifact among the candidate pace pulses by analyzing the second ECG data in view of the additional channel.

Various embodiments of the present disclosure will be described in detail with reference to the drawings, wherein like reference numerals represent like parts and assemblies throughout the several views. Additionally, any examples set forth in this specification are not intended to be limiting and merely set forth some of the many possible embodiments.

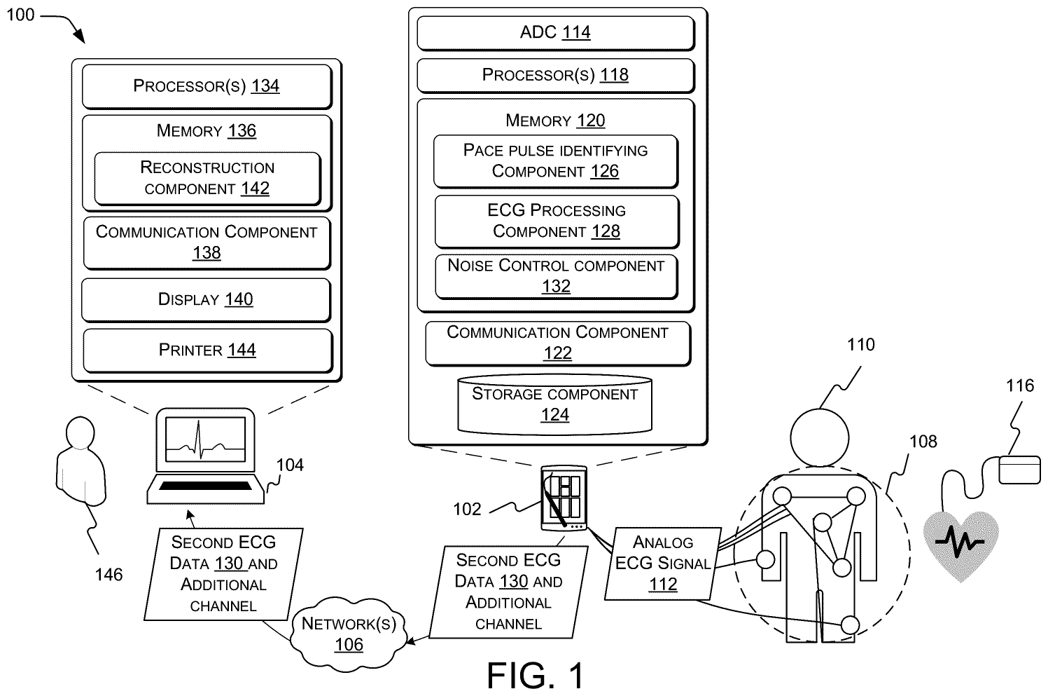

FIG. 1 shows a schematic block diagram of an example ECG system environment 100 in accordance with implementations of this disclosure. As shown in FIG. 1, the example ECG system environment 100 includes a frontend device 102 and a backend device 104. The frontend device 102 and the backend device 104 are in communication with each other, for example, via one or more networks 106. The frontend device 102 can be configured to collect, aggregate, process, and/or transmit ECG data. The backend device 104 can be configured to receive, process, print, and/or display ECG data. The networks 106 can be any type of wireless network or other communication network known in the art. Examples of the networks 106 include the Internet, an intranet, a wide area network (WAN), a local area network (LAN), and a virtual private network (VPN), cellular network connections, and connections made using protocols such as Institute of Electrical and Electronics Engineers (IEEE) standards, including 802.11a, b, g, n, and/or ac. The 802.11a, b, g, n, and/or ac are IEEE standards used for wireless routers, Wi-Fi access points, and Wi-Fi in portable devices.

The frontend device 102 includes, or is connected to, ECG sensors 108 configured to detect an analog ECG signal (such as relative voltages between electrodes) associated with a patient 110 (e.g., an animal, such as a human, with a heart). For instance, the ECG sensors 108 include electrodes that are disposed on and/or affixed to the patient 110's body. For example, the ECG sensors 108 are attached to different portions (such as the chest, the abdomen, shoulders, arms, legs, ankles, wrists, and the like) of the body of the patient 110. The ECG sensors 108 can detect differences in voltage over time across different combinations of the electrodes. These relative voltages over time are referred to as “leads.” In some examples, the ECG sensors 108 can be configured to sense a 12-lead ECG signal associated with the patient 110.

The frontend device 102 includes an analog-to-digital converter (ADC) 114 that is configured to convert relative voltages detected between the electrodes into digital data at a first sampling frequency. For example, the ADC 114 can convert the analog ECG signal 112 to first ECG data at the first sampling frequency. In some examples, the first sampling frequency can be between 8 kHz and 40 kHz (including the edges of the range). Note that the first sampling frequency is not limited to such a range, but can be any frequency range suitable for detecting candidate pace pulses in the analog ECG signal 112.

The patient 110, in various examples, may be associated with a pacemaker 116. For example, the pacemaker 116 may be implanted in the patient 110. In some implementations, the pacemaker 116 is configured to output electrical pulses to the heart of the patient 110. These electrical pulses may be referred to herein as “pace pulses.” For example, the pace pulses may be output at a frequency that increases the heart rate of the patient 110. In various implementations, the ECG sensors 108 are configured to detect the pace pulses. For instance, the analog ECG signal 112 generated by the ECG sensors 108 may indicate both the electrical activity of the heart of the patient 110 as well as the pace pulses output by the pacemaker 116.

As discussed herein, the pacemaker 116 may be configured to treat heart conditions. The pacemaker 116 can provide an external stimulus current to a region in the myocardium surrounding a pace electrode. In various examples, the stimulus current increases the membrane voltage of cells in the heart above a critical threshold for a fraction of a millisecond, at which point sodium channels in the cell membranes begin to open and an action potential sequence begins. Neighboring cells are depolarized through gap junctions, for instance, and the rest of an electrophysiology process completes the depolarization and repolarization of the ventricular tissue. The bulk of the sodium current influx completes within around five milliseconds after the external stimulus, for instance. The action potential typically takes a few hundred milliseconds, but the exact time may depend on the heart rate of the patient 110, drugs administered to the patient 110, and ion channel expression. For example, after the stimulus pulse, a pacemaker 116 can deliver a recovery pulse to avoid long-term charge build-up of the tissue. This pulse is lower in maximum amplitude than the depolarizing pulse, and may have less of an effect on the electrophysiology of the patient 110 than the depolarizing pulse.

In some examples, the frontend device 102 is associated with an analog bandwidth that is sufficient to detect candidate pace pulses in the first ECG data generated by the ADC 114. As used herein, the term “candidate pace pulse,” and its equivalents, may refer to pace pulses and/or artifact with similar frequency characteristics to pace pulses. For instance, an upper cutoff frequency of the ADC 114 of the frontend device 102 (e.g., corner frequency of the bandwidth) is at least twice a frequency associated with the pace pulses output by the pacemaker 116. In some examples, the frontend device 102 can detect pulses as short as 0.2 milliseconds (in some instances, 0.1 milliseconds). Candidate pace pulses, in some cases, are approximately rectangular. The frequency components of the candidate pace pulses, therefore, include the pulse frequency and higher harmonics of the pulse frequency, due to the rectangular windowing function (e.g., a sinc function in frequency space). However, most of the energy of a given candidate pace pulse is in the first few (e.g., one, two, or three) harmonics. As an example, a sampling frequency of 40 kHz of the ADC 114 is adequate to detect candidate pace pulses that are as short as 0.1 milliseconds.

The frontend device 102 detects the candidate pace pulses in the first ECG data using one or more processors 118 and memory 120. The processor(s) 118 can be a single processing unit or a number of units, each of which could include multiple different processing units. The processor(s) 118 can include a microprocessor, a microcomputer, a microcontroller, a digital signal processor, a central processing unit (CPU), a graphics processing unit (GPU), a security processor, etc. Alternatively, or in addition, some or all of the techniques described herein can be performed, at least in part, by one or more hardware logic components. For example, and without limitation, illustrative types of hardware logic components that can be used include a Field-Programmable Gate Array (FPGA), an Application-Specific Integrated Circuit (ASIC), an Application-Specific Standard Products (ASSP), a state machine, a Complex Programmable Logic Device (CPLD), pulse counters, resistor/coil readers, other logic circuitry, a system on chip (SoC), and/or any other devices that perform operations based on instructions. Among other capabilities, the processor(s) 118 can be configured to fetch and execute computer-readable instructions stored in the memory 120.

The memory 120 can include one or a combination of computer-readable media. As used herein, “computer-readable media” includes computer storage media and communication media. Computer storage media includes volatile and non-volatile, removable, and non-removable media implemented in any method or technology for storage of information, such as computer-readable instructions, data structures, program modules, or other data. Computer storage media includes, but is not limited to, Phase Change Memory (PRAM), Static Random-Access Memory (SRAM), Dynamic Random-Access Memory (DRAM), other types of Random-Access Memory (RAM), Read Only Memory (ROM), Electrically Erasable Programmable ROM (EEPROM), flash memory or other memory technology, Compact Disk ROM (CD-ROM), Digital Versatile Discs (DVD) or other optical storage, magnetic cassettes, magnetic tape, magnetic disk storage or other magnetic storage devices, or any other medium that can be used to store information for access by a computing device. In contrast, communication media includes computer-readable instructions, data structures, program modules, or other data in a modulated data signal, such as a carrier wave. As defined herein, computer storage media does not include communication media. In some examples, non-transitory computer-readable media does not include communication media. In some examples, the memory 120 can include an operating system configured to manage hardware and services within and coupled to a device for the benefit of other modules, components, and devices.

The frontend device 102 can include a communication component 122 configured to communicate with other computing device(s) (e.g., in mesh network) and/or to communicate via the network(s) 106. In some examples, the communication component 122 can transmit data using one or more protocols or languages, such as an extensible markup language (XML), Modbus, Hypertext Transfer Protocol (HTTP), Hypertext Transfer Protocol Secure (HTTPS), Universal Serial Bus (USB), etc.

The frontend device 102 can include a storage component 124 configured to store data, such as the first ECG data. The storage component 124 can include one or a combination of computer-readable media. For instance, the computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, hard drives, USB flash drives, secure digital (SD) cards, solid-state drives (SSD), or any other medium which can be used to store the desired information.

The memory 120 can include a pace pulse identifying component 126 which can include computer-readable instructions configured to cause the processor(s) 118 to identify one or more candidate pace pulses in the first ECG data. Features of a candidate pace pulse may include the amplitude and width of the depolarizing pulse, and the time and amplitude and/or shape of the recovery pulse. Various factors can impact the amplitude, such as the placement of electrodes of the pacemaker 116 on the heart of the patient 110 (epicardium vs. endocardium, septal vs. apex, etc.), ECG electrode placement, patient size, and pacemaker 116 model and settings, and the like. In implementations, the pace pulses may have some similarities, for example, a fast depolarizing pulse followed by a recovery pulse.

In some examples, upon executing instructions in the pace pulse identifying component 126, the processor(s) 118 can identify the candidate pace pulses in the first ECG data based on spectral signatures. The candidate pace pulses may have a similar (e.g., frequency-domain) shape to a predetermined signature pace pulse. Pace pulses made by different types of pacemakers can be different in terms of shape, width, amplitude, etc. The signature pace pulse data can indicate the shape, width, and/or the amplitude of a pace pulse output by a particular type of pacemaker. In some examples, the signature pace pulses can be provided by a third party, such as a manufacturer of the pacemaker 116, an ECG database, or the like. In some examples, the one or more candidate pace pulses can include various types of pace pulses such as atrial pace pulses, ventricular pace pules, biventricular pace pules, and so on.

The pace pulse identifying component 126 can include computer-readable instructions that cause the processor(s) 118 to generate additional data that includes waveform descriptors characterizing the candidate pace pulses. As used herein, the term “waveform descriptor,” and its equivalents, may refer to at least one characteristic of a candidate pace pulse in ECG data. In some examples, the waveform descriptors can include a segment of the first ECG data including the candidate pace pulses, compressed versions of the segments, or another type of representation of the segments, such as piecewise splines that can accurately represent the candidate pace pulse. A segment, for instance, can begin within 1, 10, or 100 milliseconds before the beginning of a given candidate pace pulse, and may end within 1, 10, or 100 milliseconds after the end of the given candidate pace pulse. For example, the pace pulse identifying component 126 can include computer-readable instructions that cause the processor(s) 118 to identify a portion of the first ECG data including a candidate pace pulse, and generate a waveform descriptor based on the portion of the first ECG data. In some examples, the waveform descriptors characterize the candidate pace pulses in any of various aspects such as the presence of the candidate pace pulses, the timing of the candidate pace pulses, shapes of the candidate pace pulses, frequency components of the candidate pace pulses, amplitudes of the candidate pace pulses, segments of the first ECG data including the candidate pace pulses, down-sampled segments of the first ECG data including the candidate pace pulses, compressed segments of the first ECG including the candidate pace pulses, or any combination thereof. In various implementations, the processor(s) 118 generate a channel based on the waveform descriptors of the candidate pace pulses. This channel may be referred to as the “additional channel,” the “pace pulse channel,” or “additional data.”

The memory 120 can include an ECG processing component 128 which can include computer-readable instructions that cause the processor(s) 118 to generate second ECG data 130 based on the first ECG data. In some instances, the high-fidelity first ECG data may require significant resources (such as storage space and bandwidth) to store, display, and/or transmit in real-time or near real-time. Furthermore, the first ECG data may be at a higher-than-necessary sampling rate for one or more ECG analyses, such as QRS complex or arrhythmia detection. Therefore, the ECG processing component 128 can process the first ECG data to improve efficiency before passing the ECG data to additional components or devices. The first ECG data can be processed in various ways. In some examples, the ECG processing component 128 can down-sample the first ECG data at a second sampling frequency to generate the second ECG data 130. In some cases, the second ECG data 130 is a compressed version of the first ECG data.

The second ECG data 130, for instance, is associated with a second sampling frequency. In some cases, the second sampling frequency is lower than the first sampling frequency. For example, the second sampling frequency is between 500 Hz and 1000 Hz (including the edges of the range). Note that the range of the second sampling frequency is not limited to such a range, but can be any frequency range suitable for clinical use of the ECG from the down-sample of the first ECG data. In some examples, the second sampling frequency can be greater than or equal to 150 Hz (for adults) or greater than or equal to 250 Hz (for children), as indicated in Kligfeld et al., 115 CIRCULATION 10, pp. 1306-24 (2007).

The second sampling frequency of the second ECG data 130 may be sufficient to identify other non-pace pulse characteristics, such as other clinically relevant ECG features. In some examples, the spectral content of the pace pulses may be in a significantly higher frequency range than the spectral content of other clinically relevant ECG features making it difficult or impossible detect certain candidate pace pulses with second sampling frequency data. An example of a non-pace pulse characteristic includes QRS complexes. In pediatric patients, for instance, a second sampling frequency between 500 Hz and 1000 Hz may be sufficient to identify QRS complexes in pediatric patients. In some examples, the second ECG data 130 has a lower cutoff frequency of about zero, in order to not distort T waves or ST segments after QRS complexes in the second ECG data 130. In some examples, the lower cut cutoff frequency can be 0.05 to 0.67 Hz. Note that such frequency ranges are examples rather than limitations.

The ECG processing component 128 can include computer-readable instructions that cause the processor(s) 118 to process the additional data comprising waveform descriptors characterizing the candidate pace pulses. For example, the ECG processing component 128 can include computer-readable instructions that cause the processor(s) 118 to down-sample portions of the first ECG data including the candidate pace pulses at a third sampling frequency. In some examples, the third sampling frequency is higher than the second sampling frequency and lower than the first sampling frequency. The waveform descriptors in the additional channel, for instance, may include the down-sampled segments of the first ECG data.

In implementations, the ECG processing component 128 can implement a filtering stage in which the data is passed through one or more filters and/or calculators. For example, the filtering stage can include a first difference, an 8-tap moving average filter (implemented with a single sum and difference), error accumulator calculation (sum), and quantization (shift). In some examples, the ECG processing component 128 can include computer-readable instructions that cause the processor(s) 118 to use one or more compression algorithms to reduce the data amount. In some examples, the ECG processing component 128 can compress the additional pace related data from the first ECG data after generating the second ECG data 130. In some examples, compression algorithms can use Gzip to perform compression. Gzip is a file format and software application used for file compression and decompression. Gzip was developed by GNU Project, and was initially released on 31 Oct. 1992. Note that other compression algorithms suitable for compressing the first ECG data and/or the additional data can be used.

The memory 120 can include a noise control component 132, which can include computer-readable instructions that cause the processor(s) 118 to mitigate the noise in the first ECG data, the additional channel, and/or the second ECG data 130. In some instances, the noise control component 132 can reduce the noise in the first ECG data and/or the additional data before the second ECG data 130 is generated. In some instances, the noise control component 132 can include computer-readable instructions that cause the processor(s) 118 to mitigate noise storms, which may refer to a situation where a number of noise signals occur within a relatively short period. In some examples, when a number of consecutive spikes occur in the first ECG data, the noise control component 132 can disable the transmission of the corresponding second ECG data 130 for a period of time. As an example, the noise control component 132 can disable the data transmission for a few hundred milliseconds when five consecutive spikes narrower than 350 microseconds (usec) occur within a short time (e.g., 500 milliseconds).

In some examples, the frontend device 102 can at least temporarily transmit the first ECG data to the backend device 104 without down-sampling or compression as long as there are sufficient resources for the backend device 104 to output the first ECG data in real-time or near-real-time. For example, the frontend device 102 may have sufficient computing power and bandwidth to process and transmit high-resolution ECG data. As such, the frontend device 102 does not need to down-sample or compress the high-resolution first ECG data and can transmit the first ECG data to the backend device 104 without reducing the data amount.

The frontend device 102 can send the second ECG data 130 and the additional channel to the backend device 104 via the network(s) 106. In turn, the backend device 104 can receive the second ECG data 130 and the additional channel from the frontend device 102 via the networks(s) 106. The backend device 104 includes one or more processor(s) 134, a memory 136, and a communication component 138, each of which can be implemented similar to the processor(s) 118, the memory 120, and/or the communication component 122 of the frontend device 102.

The backend device 104 may further include a display 140 configured to visually output one or more ECG waveforms based on the second ECG data 130. For example, the backend device 104 may include a display (e.g., a screen) that visually outputs one or more waveforms respectively corresponding to the leads represented by the second ECG data 130. In some cases, the backend device 104 includes a printer 144 that can print the ECG waveform(s) on a substrate (e.g., paper, plastic sheets, cloth, or the like). According to various implementations, the backend device 104 outputs the waveform(s) in real-time or near-real-time, such that there is minimal latency between the detected ECG and the waveforms output by the backend device 104. For example, the backend device 104 may output a lead voltage within a period of time such as 1 millisecond (ms) of the voltage being detected by the ECG sensors 108. In some examples, the backend device 104 can print an ECG waveform in response to the user's 146 input (e.g., by pressing a “print” button). In some examples, the backend device 104 can print the ECG waveform(s) continuously.

In addition, the backend device 104 further outputs the additional channel. For example, the display 140 or printer 144 may visually output dashes overlaying the ECG waveform(s) indicating the timing of the detected candidate pace pulses. In some cases, the user 146 may operate the backend device 104 to zoom in on a portion of an ECG waveform including a candidate pace pulse, such that the display 140 or the printer 144 may view a higher-fidelity version of the candidate pace pulse included in the additional channel, rather than the lower-fidelity ECG represented in the second ECG data 130. Other ways of conveying the waveform descriptors are described herein.

The memory 136 can include a reconstruction component 142 which can include computer-readable instructions that cause the processor(s) 134 to reconstruct ECG waveforms based on the second ECG data 130, as well as the waveform descriptors characterizing one or more candidate pace pulses from the additional data. As discussed herein, the first ECG data and/or the second ECG data can be 12-lead ECG data. A 12-lead ECG can include 12 waveforms. Note that the ECG waveforms can be utilized, corresponding to a 12-lead ECG, but implementations are not so limited.

In some cases, the backend device 104 further distinguishes true pace pulses from artifact among the candidate pace pulses represented in the additional channel. For instance, the reconstruction component 142 can include computer-readable instructions that cause the processor(s) 134 to identify one or more artifacts among the candidate pace pulses in the additional data, which includes waveform descriptors characterizing the candidate pace pulses. Artifacts may refer to portions of the ECG and/or ECG data that are not related to cardiac electrical activity or pace pulses. In some instances, artifacts can be caused by line currents, tremors, muscle shivering, hiccups, interference from other medical devices, and the like.

In some instances, paced ventricular beats can have some similarities with signals associated with a left bundle branch block (LBBB), which is a delay or blockage of electrical impulses to the left side of the heart. For example, paced beats can be stimulated near the apex (if the pacemaker is implanted there). In the case of LBBB signals, the left ventricular depolarization can be stimulated up through the apex after traveling down the (much smaller) right ventricle. In some instances, LBBB can also produce notched QRS complexes that can mimic paced fusion beats. If the pace pulses are tiny or impossible to discern, it can be difficult to differentiate between the two (LBBB or ventricular pacing).

In some instances, techniques such as His bundle pacing (HBP) or septal pacing may lead to narrow-QRS paced rhythms. His bundle pacing (HBP) is an alternative approach to right ventricular (RV) and biventricular pacing and is performed with the aim of maintaining a physiological pattern of ventricular activation via the native His-Purkinje system.

In some cases, the backend device 104 can identify paced rhythms by analyzing the additional channel. In some instances, intermittent pacing can occur, and may be difficult to interpret why it occurs. Intermittent pacing may refer to a situation where a paced beat occurs, followed by a long pause, and then the pacing resumes. In some examples, recognizing intermittent pacing can be clinically useful. Optionally, the backend device 104 notifies the user 146 of the intermittent pacing. In some instances, intermittent pacing may be because normally conducted beats are detected by the pacemaker. If the pacemaker is not outputting pace pulses at regular intervals, it can be harder to differentiate between real pace pulses and interference, if the sampling frequency is 1000 Hz or below. In some instances, pacemaker non-capture where the myocardium does not respond to the electrical stimuli from the pacemaker, can be dangerous for the patient. In some instances, in the case of cardiac resynchronization therapy (CRT), at a sampling frequency of 1000 Hz or lower, it can be hard to tell the difference between biventricular pacing and single-ventricle pacing.

The reconstruction component 142 can include computer-readable instructions that cause the processor(s) 134 to identify/classify a candidate pace pulse in the first ECG data as an artifact (rather than a candidate pace pulse), if the candidate pace pulse is not followed by paced rhythm. In some examples, upon executing instructions in the reconstruction component 142, the processor(s) 134 can reject/remove the identified artifacts from the first ECG data. Additionally or alternatively, the reconstruction component 142 can include computer-readable instructions that cause the processor(s) 134 to identify the artifacts without removing the identified artifacts from the first ECG data. Instead, the system can allow the backend device 104 to present the identified artifacts with artifacts indicators for review by the user 146.

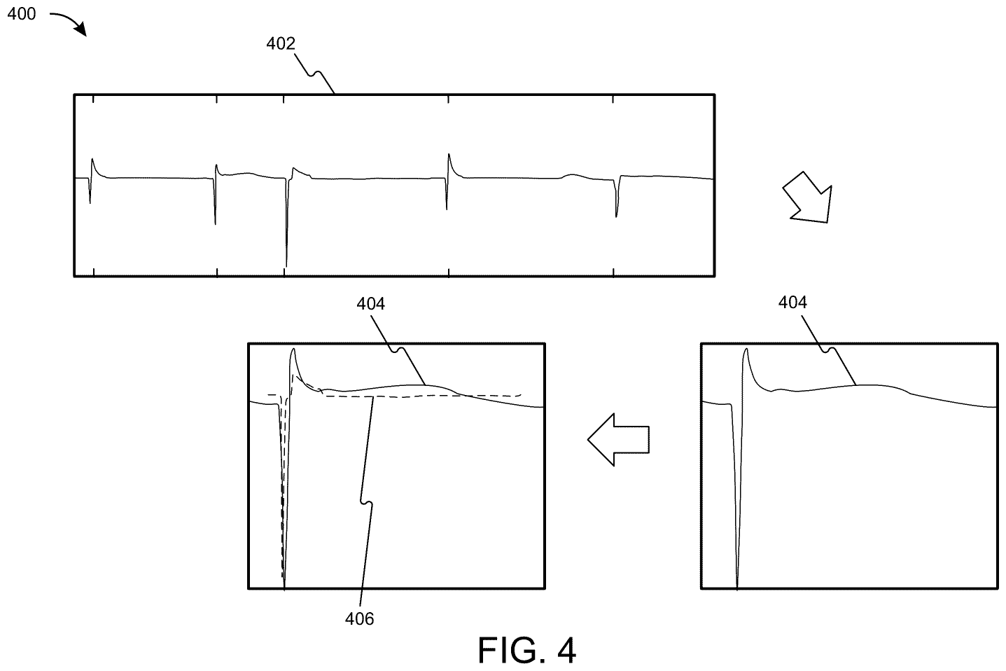

The reconstruction component 142 can include computer-readable instructions that cause the processor(s) 134 to generate an average pace pulse by performing signal averaging on the candidate pace pules. The reconstruction component 142 can include computer-readable instructions that cause the processor(s) 134 to compare the average pace pulse to an individual candidate pace pulse, and determine a discrepancy between the average pace pulse and the individual candidate pace pulse. As described herein, the discrepancy can refer to the mismatch between two pace pulses in terms of the shape, the amplitude, the frequency, etc. In some cases, the discrepancy is the inverse of the similarity between two waveforms. For example, the discrepancy between a candidate pace pulse and an average pace pulse may refer to an inverse of a cross-correlation between the candidate pace pulse and the average pace pulse. The reconstruction component 142 can include computer-readable instructions that cause the processor(s) 134 to determine that the discrepancy is greater than a threshold, and based on determining that the discrepancy is greater than the threshold, modify the additional data by removing the individual candidate pace pulse. Additional details are given throughout this disclosure, for example, with respect to FIG. 4.

In some embodiments, the display 140 can display a user interface (UI) which may facilitate an interaction between the backend device 104 and the user 146. The display 140 can present the ECG waveform(s) and waveform descriptors characterizing the candidate pace pulses together or separately. As discussed herein, the second ECG data 130 can include 12-lead ECG data. For example, the display 140 can present twelve ECG waveforms corresponding to twelve leads, and present the waveform descriptors characterizing one or more candidate pace pulses in a separate user interface element (e.g., a separate waveform) that is displayed outside of the twelve ECG waveforms. As an example, the waveform descriptors in the additional data can be represented by a rhythm strip that is output next to the ECG waveforms on the display 140, wherein the rhythm strip can share a time scale with the ECG waveforms. As described herein, ECG can capture the electrical activity of the heart. A rhythm strip may refer to tracing within a period, showing the heart activity from one or more leads. The waveform descriptor can characterize the candidate pace pulses in terms of frequency, amplitude, shape, or the like.

Additionally or alternatively, the display 140 can present the ECG waveforms in twelve leads, and present the waveform descriptors within the twelve leads. For example, the waveform descriptors can include vertical lines above and/or below the ECG waveforms indicating the presence and timing of the one or more candidate pace pulses. As another example, the waveform descriptors can include vertical lines superimposed on the ECG waveforms, indicating the presence and timing of the one or more candidate pace pulses.

As described herein, there can be artifacts among the candidate pace pulses. In some examples, there can be no artifact in the ECG waveforms. If there are artifacts, the display 140 can present artifact indicators in the ECG waveforms, indicating the presence of one or more artifacts.

The display 140 can present the one or more candidate pace pulses with a detected (e.g., as-detected by the frontend device 102 in the analog ECG signal 112) amplitude without increasing the amplitude of the one or more candidate pace pulses. As discussed herein, in some instances, the candidate pace pulses can be presented (e.g., displayed and/or printed out) as spikes with an increased voltage amplitude in an example ECG waveform, to emphasize the presence of the candidate pace pulses and aid recognition by the user 146. However, the increased amplitude of the candidate pace pulses in this method of display may not be proportional to the voltage amplitude as detected by the ECG sensors 108, making the spikes inaccurate. Therefore, it may be favorable to display the candidate pace pulses with an accurate voltage amplitude.

The user 146 (e.g., a cardiologist, a physician, or the like) can view the ECG waveforms and/or the waveform descriptors characterizing the candidate pace pulses presented via the user interface on the display 140 of the backend device 104. In some examples, the user 146 can view the ECG waveforms and/or the waveform descriptors characterizing the candidate pace pulses printed out on a substrate (e.g., paper, plastic sheets, cloth, or the like) or any other suitable media. In some examples, the display 140 can display the user interface that allows the user 146 to zoom in on the ECG waveforms and/or the waveform descriptors via the user interface. For example, the display 140 can allow, via the user interface, the user 146 to zoom in where candidate pace pulses are present, such that the user 146 can take a closer look at the candidate pace pulses. The user 146, for instance, may manually determine whether a candidate pace pulse is a true pace pulse or an artifact based on viewing the zoomed-in version of the candidate pace pulse.

Moreover, the backend device 104 can determine the various statuses of the second ECG data 130 by implementing ECG interpretation algorithms. The ECG interpretation algorithms can be configured to generate interpretation statements and so on. For example, the backend device 104 can implement ECG interpretation algorithms to determine that pacemaker activity is detected but a paced rhythm is not (indicating that unspecified pacemaker activity is detected); a high-frequency artifact detected that resembles pacemaker pulses on the surface ECG at the second sampling frequency but not at the first sampling frequency (indicating that high-frequency interference is detected); biventricular pacemaker activity is detected; and so on. As such, the display 140 can present one or more statements for user 146's consideration, such as “Pacemaker activity is detected but a paced rhythm is not (e.g., with non-capture),” “High-frequency artifact detected that resembles pacemaker pulses,” “Biventricular pacemaker detected,” or the like.

Additionally, the additional data including the waveform descriptors may be stored in a database (e.g., a cloud database, an online database, a local database, a remote database, and the like), allowing further analysis and research to be conducted. For example, algorithms that can identify/classify pacemaker 116 more accurately can be developed, such as algorithms that can identify the manufacturer or the brand of the pacemaker 116 based on the depolarizing and recovery pulse “signatures.”

The techniques described herein may be performed by any of the frontend device 102, the backend device 104, and/or other devices or components not explicitly shown or described. For example, other devices that are contemplated that may be used to implement the described techniques include (but are not limited to) bedside monitors, Holter recorders, stress test systems, and so forth.

Although FIG. 1 illustrates the frontend device 102 and the backend device 104 as separate devices, implementations are not so limited. For example, the various components of the frontend device 102 and the backend device 104 may be integrated into a single device. In some cases, the network(s) 106 can be omitted, such that the frontend device 102 provides the second ECG data 130 to the backend device 104 over one or more wired connections within the single, integrated device.

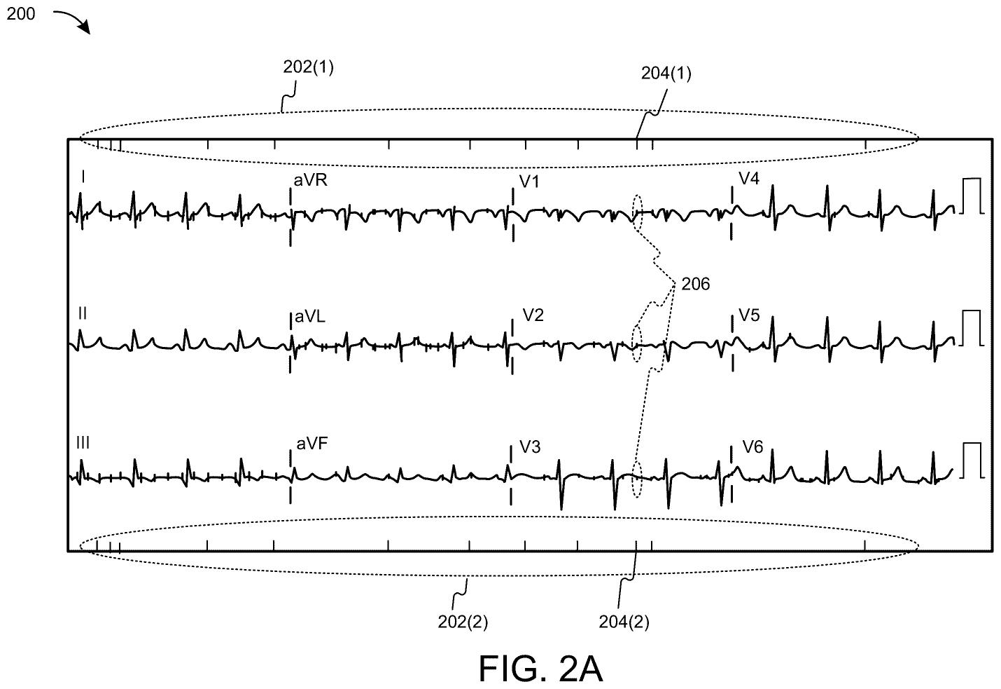

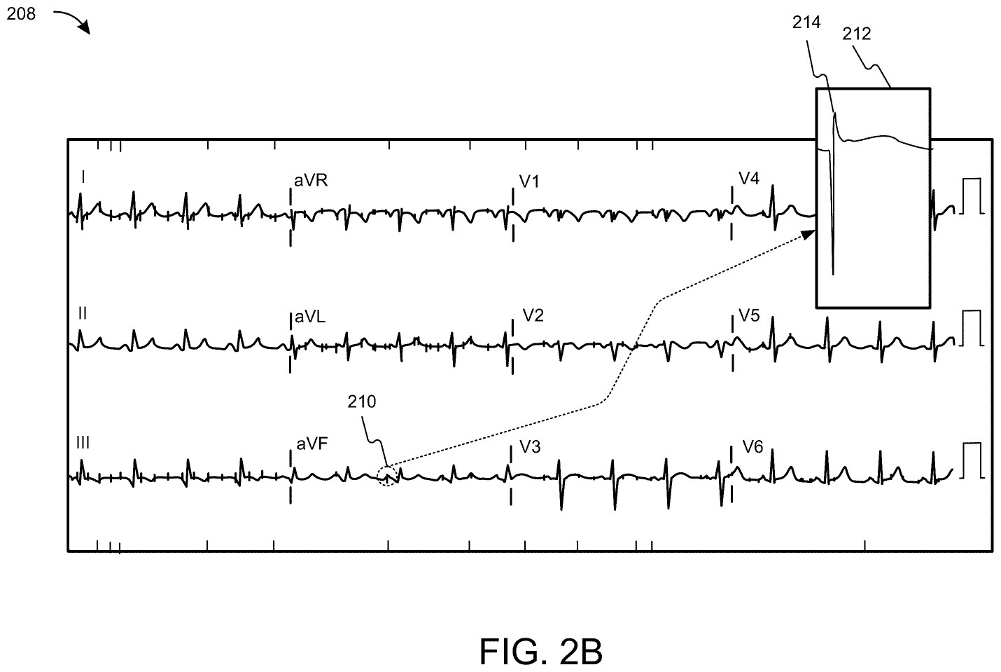

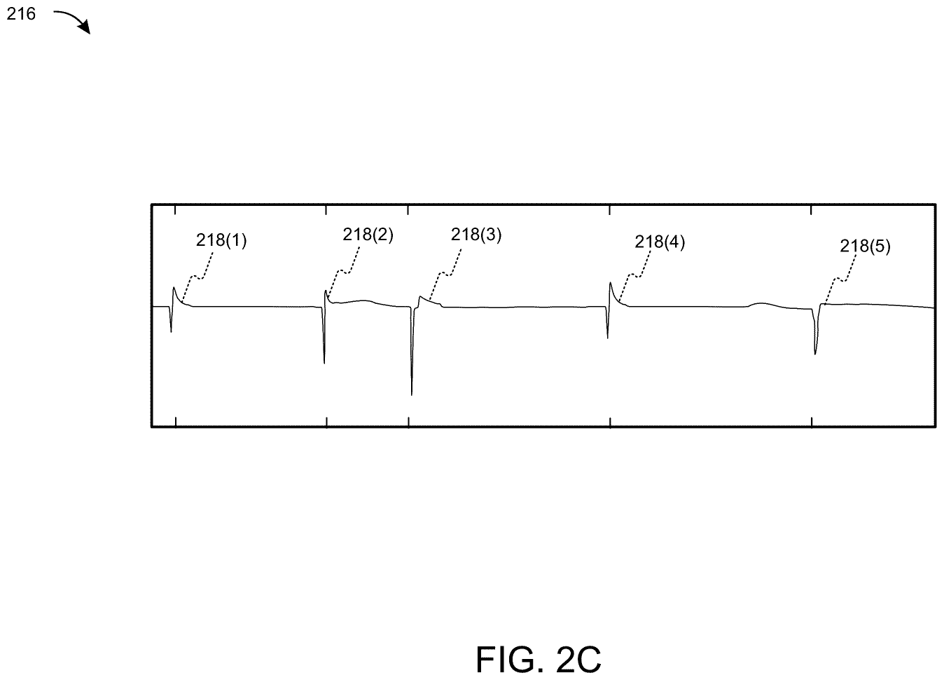

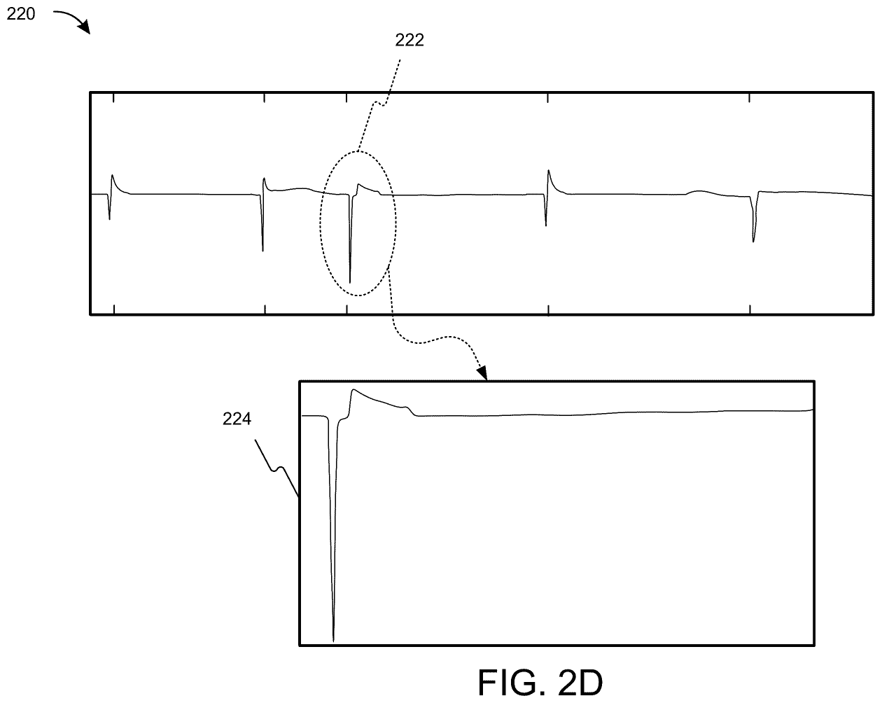

FIG. 2A illustrates example ECG waveforms 200 in accordance with implementations of this disclosure. The example ECG waveforms 200 can be displayed via the display 140 or printed out by the printer 144 as described with respect to FIG. 1. As shown in FIG. 2A, the example ECG waveforms 200 can include twelve leads, for example, three bipolar limb leads (I, II, and III), the unipolar limb leads (aVR, aVL, and aVF), and six unipolar chest leads, also called precordial or V leads, (V1, V2, V3, V4, V5, and V6). AVR means augmented vector right, where a positive electrode can be on the right shoulder of the patient. AVL means augmented vector left, where the positive electrode can be on the left shoulder of the patient. AVF means augmented vector foot, where a positive electrode can be on the foot of the patient.

The example ECG waveforms 200 can include the waveform descriptors characterizing one or more candidate pace pulses. For example, waveform descriptors can include one or more pulse indicators (denoted by dashed circles 202(1) and 202(2)) can indicate the presence of the candidate pace pulses. For example, the pulse indicators can include vertical lines at the top and/or bottom of the ECG waveform 200. As an example, a pair of vertical lines such as a vertical line 204(1) at the top of ECG waveforms 200 and a corresponding vertical line 204(2) at the bottom of ECG waveforms 200 can indicate the presence of a candidate pace pulse 206. Note that the vertical lines are examples rather than limitations of the pulse indicators. The pulse indicators can be in any form (e.g., arrows, circles, color indicators, icons, and the like) suitable to indicate the presence of candidate pace pulses.

In some examples, though not shown in FIG. 2A, the pulse indicators (e.g., vertical lines and the like) can be presented superimposed on the ECG waveform 200. As described herein, there may or may not be no artifact among the candidate pace pulses. In some examples, though not shown in FIG. 2A, the ECG waveforms 200 can include artifact indicators, indicating the presence of one or more pace artifacts. For example, the artifact indicators can be different from the pulse indicators.

FIG. 2B illustrates an example scenario 208 where a portion of an ECG waveform is zoomed in in accordance with implementations of this disclosure.

As described above, the user can zoom in on a portion of the ECG waveform or a portion of the waveform descriptor conveniently. For example, an ECG system can receive an input signal indicating a zoom-in instruction from the user to zoom in a portion 210 of an ECG waveform. The zoom-in instruction can be input by a gesture, a voice instruction, a button on the keyboard, a tap on a touchpad, etc. In some examples, the ECG system can present, on a display, a zoomed-in view 212 of the portion 210 of the ECG waveform upon receiving the input signal. For example, the user can click at the portion 210 of the ECG waveform. In response to the clicking, the ECG system can present the corresponding zoomed-in view 212 of the portion 210 of the ECG waveform. As an example, a candidate pace pulse 214 that is almost invisible in the ECG waveform can be shown in the zoomed-in view 212, such that the user can conveniently view and analyze the pace pulses.

FIG. 2C illustrates an example view 216 of waveform descriptors characterizing one or more candidate pace pulses 218(1), 218(2), 218(3), 218(4), and 218(5) in accordance with implementations of this disclosure. Note that though FIG. 2C shows five candidate pace pulses 218(1), 218(2), 218(3), 218(4), and 218(5), there can be other numbers of candidate pace pulses. Moreover, the candidate pace pulses 218(1), 218(2), 218(3), 218(4), and 218(5) are shown in FIG. 2C are examples rather than limitations.

As described herein, an ECG system can present, on a display ECG waveforms and waveform descriptors characterizing one or more candidate pace pulses. In some examples, the waveform descriptors characterizing one or more candidate pace pulses can be presented separately. The example view 216 can be displayed via the display 140 or printed out by the printer 144 as described with respect to FIG. 1.

FIG. 2D illustrates an example scenario 220 where a portion of the waveform descriptors is zoomed in in accordance with implementations of this disclosure.

As described above, the user can zoom in on a portion of the waveform descriptors conveniently. For example, an ECG system can receive an input signal indicating a zoom-in instruction from the user to zoom in a portion 222 of the waveform descriptors. The zoom-in instruction can be input by a gesture, a voice instruction, a button on the keyboard, a tap on a touchpad, etc. In some examples, the ECG system can present, on a display, a zoomed-in view 224 of the portion 222 of the waveform descriptors upon receiving the input signal. As such the user can conveniently view the portion 222 of the waveform descriptors in detail.

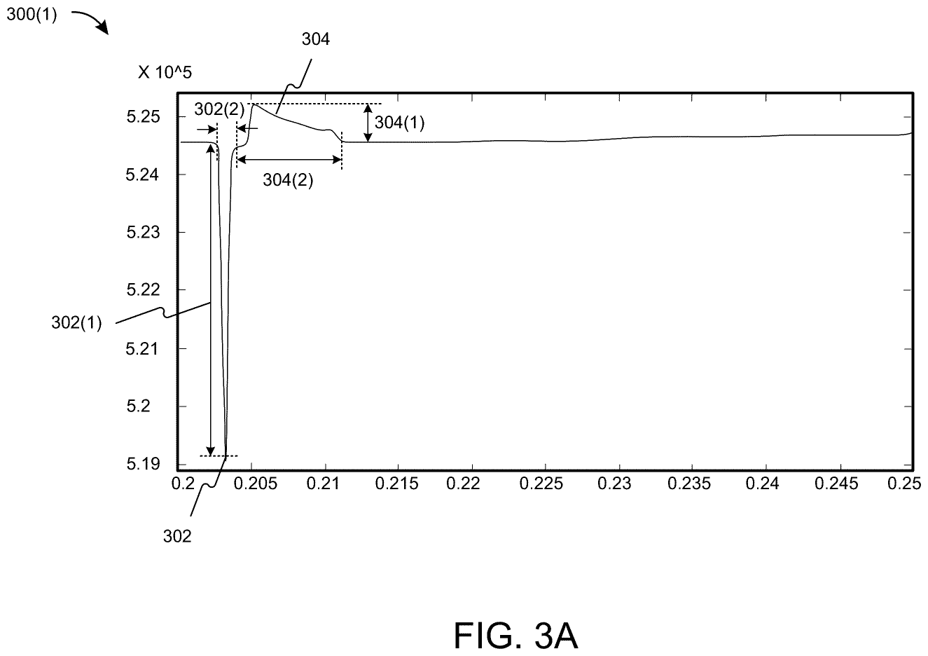

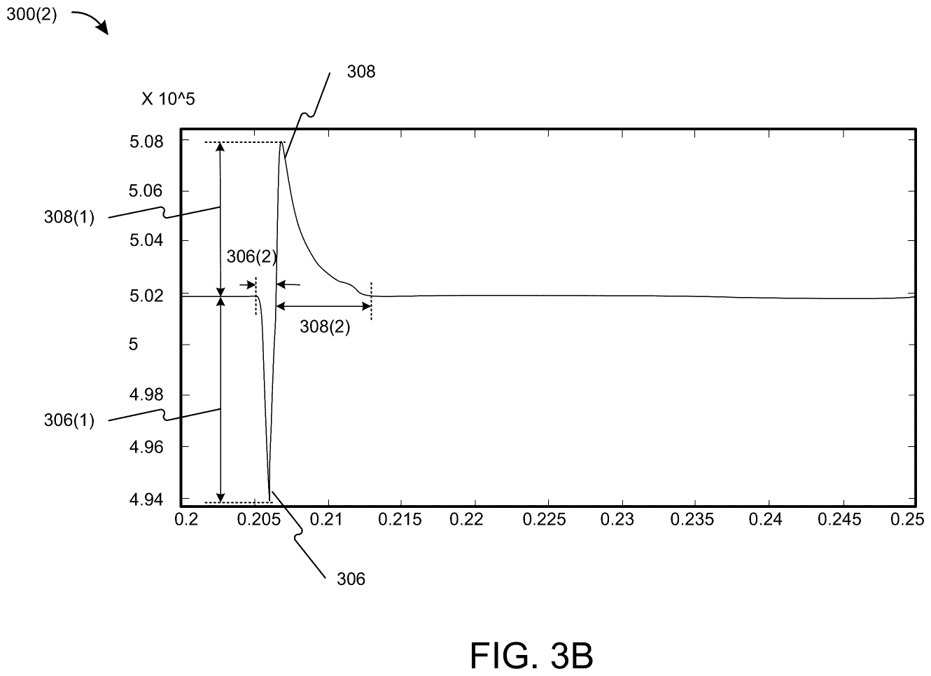

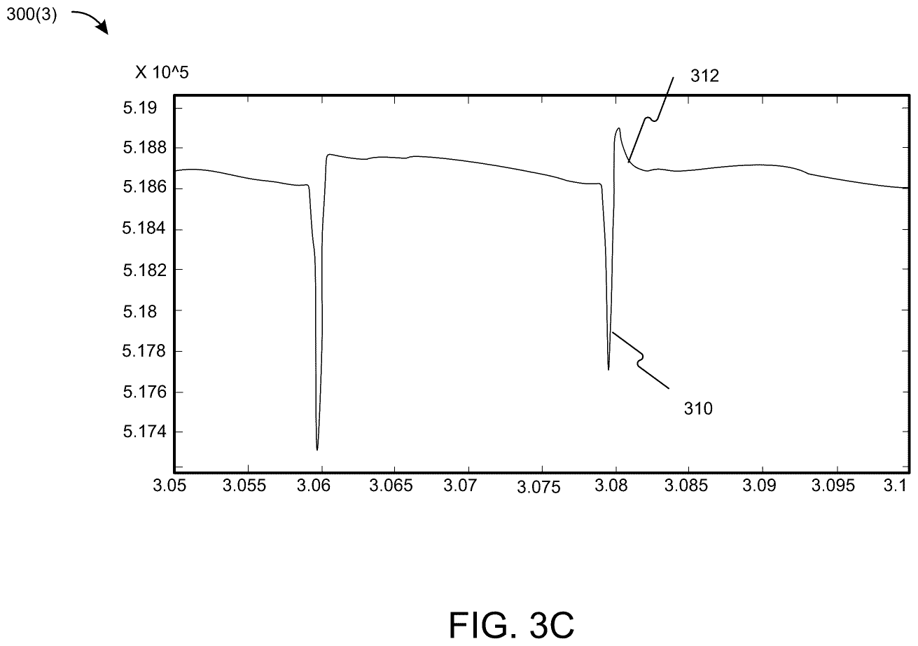

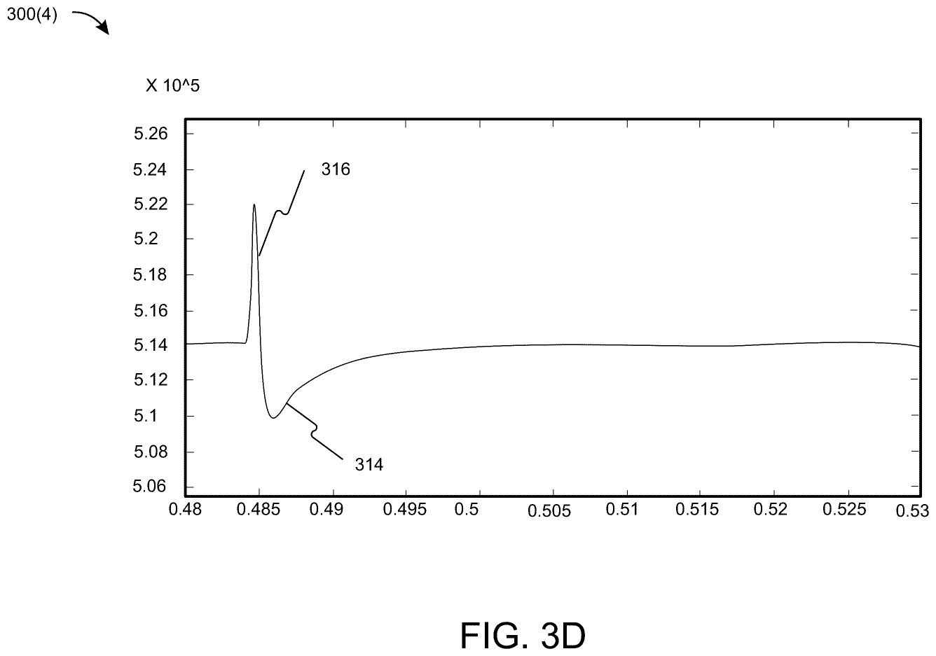

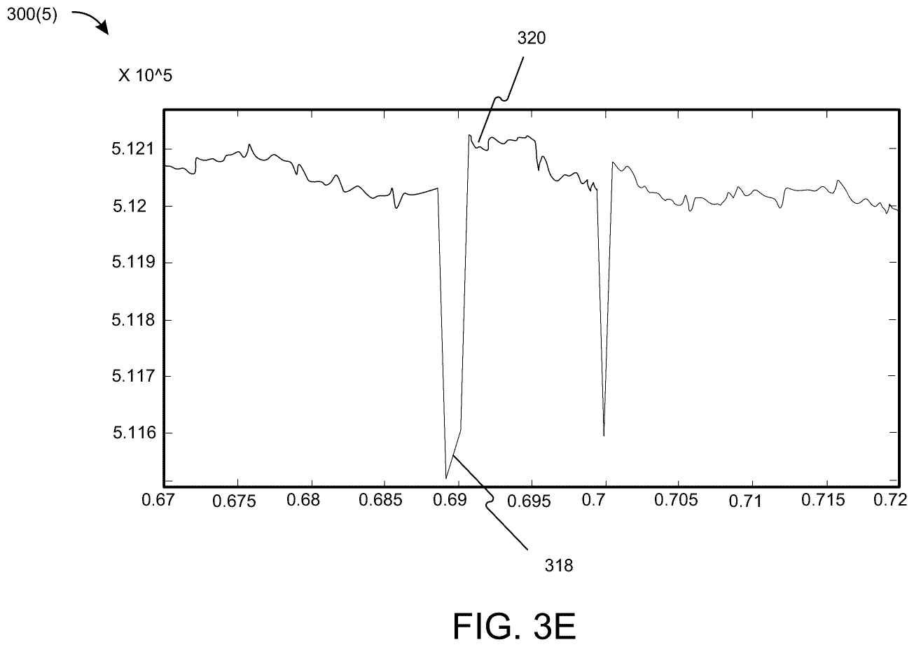

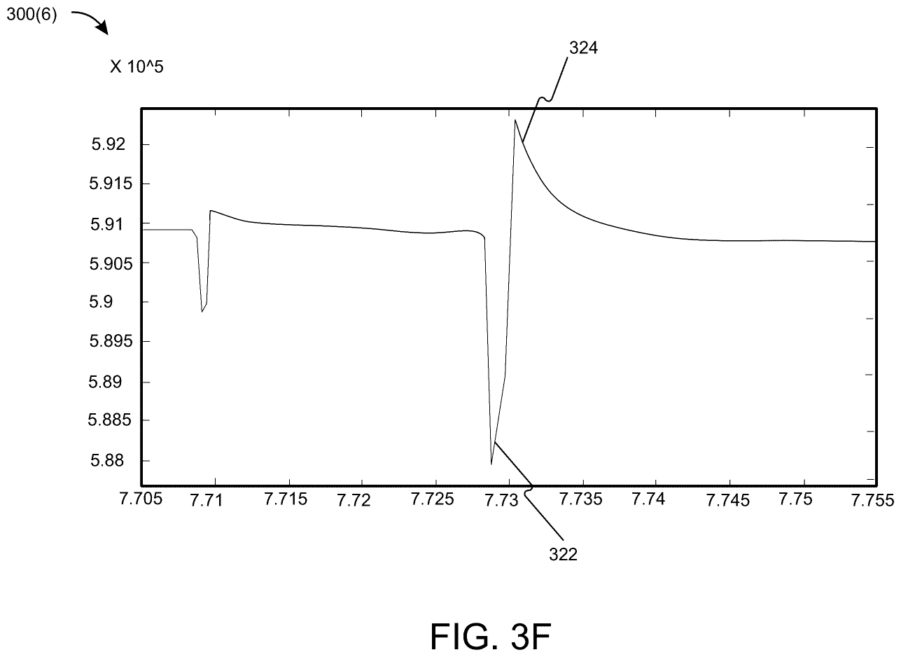

FIGS. 3A, 3B, 3C, 3D, 3E, and 3F illustrate example pace pulses in accordance with implementations of this disclosure. As described herein, the pace pulses can have various types of morphology. Pace pulses 300(1), 300(2), 300(3), 300(4), 300(5), and 300(6) are shown in FIGS. 3A, 3B, 3C, 3D, 3E, and 3F are examples of various types of morphology of pace pulses. Note that that such examples are for illustration purposes rather than limitations. In implementations, the example pace pulses 300(1), 300(2), 300(3), 300(4), 300(5), 300(6) can be a part of the ECG waveforms or waveform descriptors displayed in the display 140 or printed out by the printer 144 as described with respect to FIG. 1.

In FIGS. 3A, 3B, 3C, 3D, 3E, and 3F, the horizontal axis can represent the time in seconds, and the vertical axis can represent the LSB (least significant bit) of the frontend device (such as the frontend device 102 described with respect to FIG. 1), for instance. LSB may refer to the amplitude of one step (i.e., one digital number (DN)) from the ADC. In some instances, the ECG waveform can represent the ECG signal at 1000 Hz with an amplitude resolution of 0.9375 uV/LSB. Note that the resolution discussed here are examples rather than limitations, there can be various resolutions for different devices. The LSBs are volts times a scaling factor. In some instances, the amplitude of pace pulses (from internal pacemakers) in the ECG can be a few hundred microvolts to several millivolts. In some instances, the amplitude of pace pulses (from external pacemakers) can be up to 700 mV.

FIG. 3A illustrates an example pace pulse 300(1) in accordance with implementations of this disclosure. As shown in FIG. 3A, the pace pulse 300(1) can include a depolarizing pulse 302 followed by a recovery pulse 304. The depolarizing pulse 302 can have an amplitude 302(1) and a duration 302(2). The recovery pulse 304 can have an amplitude 304(1) and a duration 304(2).

FIG. 3B illustrates an example pace pulse 300(2) in accordance with implementations of this disclosure. As shown in FIG. 3B, the pace pulse 300(2) can include a depolarizing pulse 306 followed by a recovery pulse 308. The depolarizing pulse 306 can have an amplitude 306(1) and a duration 306(2). The recovery pulse 308 can have an amplitude 308(1) and a duration 308(2).

FIG. 3C illustrates an example pace pulse 300(3) in accordance with implementations of this disclosure. As shown in FIG. 3C, the pace pulse 300(3) can include a depolarizing pulse 310 followed by a recovery pulse 312.

FIG. 3D illustrates an example pace pulse 300(4) in accordance with implementations of this disclosure. As shown in FIG. 3D, the pace pulse 300(4) can include a depolarizing pulse 314 followed by a recovery pulse 316.

FIG. 3E illustrates an example pace pulse 300(5) in accordance with implementations of this disclosure. As shown in FIG. 3E, the pace pulse 300(5) can include a depolarizing pulse 318 followed by a recovery pulse 320.

FIG. 3E illustrates an example pace pulse 300(6) in accordance with implementations of this disclosure. As shown in FIG. 3F, the pace pulse 300(6) can include a depolarizing pulse 322 followed by a recovery pulse 324.

FIG. 4 illustrates an example scenario 400 where a comparison is made between an individual candidate pace pulse with an average pace pulse in accordance with implementations of this disclosure. Referring to FIG. 4, as described herein, waveform descriptors 402 characterizing one or more candidate pace pulses can be obtained from high-fidelity ECG data. As described herein, an ECG system according to this disclosure can perform signal averaging on the waveform descriptors 402 to generate an average pace pulse 404. Then, the average pace pulse 404 can be compared with an individual candidate pace pulse 406. In some examples, a discrepancy can be determined between the average pace pulse 404 and the individual candidate pace pulse 406. If the discrepancy is determined to be greater than a threshold, the ECG system can modify the waveform descriptors in the additional data by removing the individual candidate pace pulse. In some examples, the discrepancy can be calculated based on a sum of respective amplitude differences between the average pace pulse 404 and the individual candidate pace pulse 406 at the respective sampling time point. Additionally or alternatively, the discrepancy can be calculated based on correlation (e.g., Pearson correlation coefficient, phase correlation, etc.), morphology matching, clustering of features such as frequency component amplitudes, or the like. The threshold for the discrepancy can be determined arbitrarily or based on the user's experience.

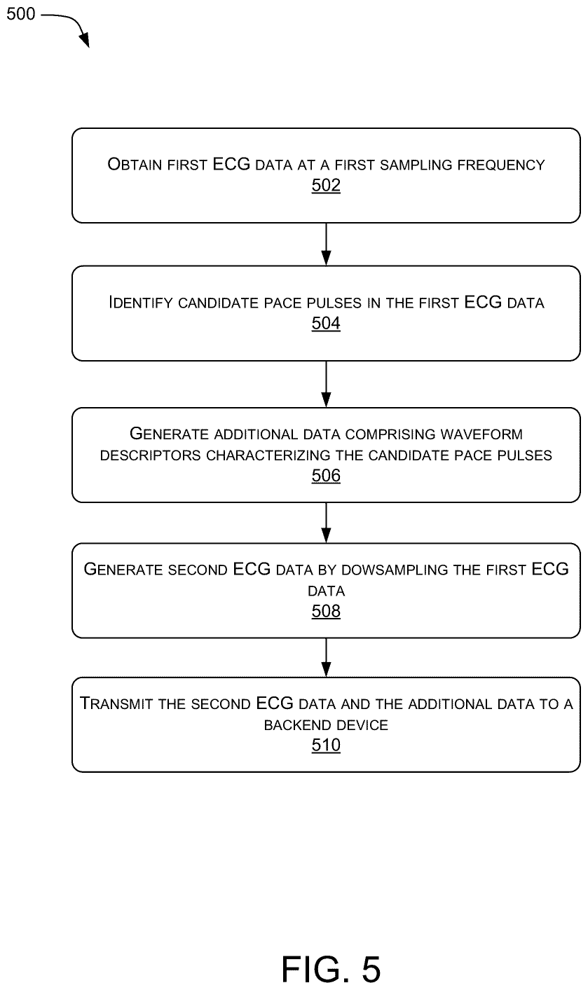

FIG. 5 illustrates a flow chart of an example process 500 for detecting pace pules in the ECG data in accordance with implementations of this disclosure. In some examples, at least some operations of the process 500 may be performed by one or more computing devices, such as the frontend device 102 described with respect to FIG. 1. Note that the operations of the process 500 are not limited to the orders presented. Rather, the operations of the process 500 can be performed in any proper order. Moreover, at least some operations of the process 500 can be repeated, combined, or split to implement techniques of this disclosure.

At operation 502, the process can include obtaining, by a frontend device, the first ECG data at a first sampling frequency. In some examples, the frontend device can obtain an analog ECG signal via multiple sensors, and convert, via an ADC, the analog ECG signal to the first ECG data at a first sampling frequency. The sensors can be placed in a configuration across a patient's body (such as the chest, abdomen, shoulders, arms, legs, ankles, wrists, and the like). Additional details are given throughout this disclosure. In some examples, the first ECG data is associated with the first sampling frequency. In some examples, the first sampling frequency can be between 8 kHz and 40 kHz (including the edges of the range). Note that the range of the first sampling frequency is not limited to such a range, but can be any frequency range suitable for detecting the candidate pace pulses.

At operation 504, the process can include identifying, by the frontend device, candidate pace pulses in the first ECG data. In some examples, the frontend device can identify the pace pulses in the first ECG data by identifying a recovery pulse. A time window around the detection time for the candidate pace pulse may be used to bound the capture of additional pace related data. In some examples, the frontend device can identify the pace pulses by detecting edges (such as rising edges, falling edges, and the like) of the pace pulse. In some examples, the frontend device can identify the candidate pace pulses in the first ECG data based on signature pace pulse data. In some examples, the signature pace pulse data can be provided by a third party, such as a pacemaker manufacturer, an ECG database, and the like. In some examples, the candidate pace pulses can include various types of pace pulses such as atrial pace pulses, ventricular pace pules, biventricular pace pules, and the like. Additional details of examples of pace pulses are given throughout this disclosure such as in FIGS. 3A-3F.

At operation 506, the process can include generating, by the frontend device, additional data comprising waveform descriptors characterizing the candidate pace pulses. In some examples, the waveform descriptors characterize the candidate pace pulses in various aspects such as the presence of the candidate pace pulses, the timing of the candidate pace pulses, shapes of the candidate pace pulses, frequency components of the candidate pace pulses, amplitudes of the candidate pace pulses, or the like. Additional details of the waveform descriptors are provided throughout this disclosure.

At operation 508, the process can include generating, by the frontend device, second ECG data by down-sampling the first ECG data. In some instances, the high-fidelity first ECG data may require a lot of resources (such as storage space and bandwidth) to store and/or transmit in real-time or near real-time. However, the frontend device may have limited resources (such as storage space and bandwidth). Therefore, the frontend device can process the first ECG data to reduce the data amount to save resources. The first ECG data can be processed in various ways. In some examples, the frontend device can down-sample the first ECG data at a second sampling frequency to generate the second ECG data. For example, the second sampling frequency is between 500 Hz and 1000 Hz (including the edges of the range). Note that the range of the second sampling frequency is not limited to such a range, but can be any frequency range suitable for processing and storing the first ECG data. In some examples, the second ECG data 130 can include a brief interval of the first ECG data, compressed samples, or a representation such as piecewise splines that can accurately represent the candidate pace pulses.

At operation 510, the process can include transmitting, by the frontend device, the second ECG data to a backend device. For example, the frontend device and the backend device are in communication with each other, via one or more networks.

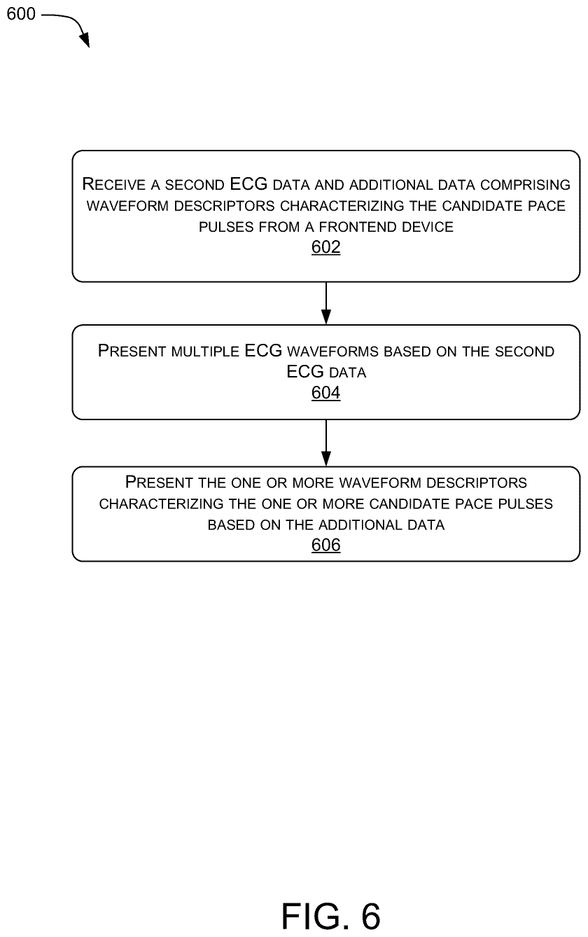

FIG. 6 illustrates a flow chart of an example process 600 for detecting pace pules in the ECG data in accordance with implementations of this disclosure. In some examples, at least some operations of the process 600 may be performed by one or more computing devices, such as the backend device 104 described with respect to FIG. 1. Note that the operations of the process 600 are not limited to the orders presented. Rather, the operations of the process 600 can be performed in any proper order. Moreover, at least some operations of the process 600 can be repeated, combined, or split to implement techniques of this disclosure.

At operation 602, the process can include receiving, by a backend device, a second ECG data and additional data comprising waveform descriptors characterizing the candidate pace pulses from a frontend device. The second ECG data can be generated based on a first ECG data associated with a first sampling frequency. The second ECG data is associated with a second sampling frequency lower than the first sampling frequency.

At operation 604, the process can include presenting, by the backend device, multiple ECG waveforms based on the second ECG data. In some examples, the waveform descriptors can characterize shapes of the candidate pace pulses. In some examples, the waveform descriptors can characterize frequency components of the candidate pace pulses. In some examples, the waveform descriptors can characterize amplitudes of the candidate pace pulses. In some examples, the waveform descriptors can include indicators (such as vertical lines at the top and/or bottom of the ECG waveform), indicating the presence and the timing of the candidate pace pulses. In some examples, the waveform descriptors can include the raw 40 kHz samples for a brief interval, compressed samples, or a representation such as piecewise splines that can accurately represent the pace pulse signal.

At operation 606, the process can include presenting, by the backend device, the one or more waveform descriptors characterizing the candidate pace pulses based on the additional data.

The backend device can present the ECG waveforms and the waveform descriptors together or separately. As discussed herein, the second ECG data can be 12-lead ECG data, such that twelve waveforms representing the twelve leads can be presented. For example, the backend device can present the waveform descriptors in a separate user interface element than the twelve waveforms. Additionally or alternatively, the backend device can present the waveform descriptors within at least one of the twelve waveforms. Note that the 12-lead ECG data is a non-limiting example, and there can be greater or fewer leads and/or channels presented according to various implementations. In some examples, the ECG waveforms and the waveform descriptors characterizing the candidate pace pulses can be printed on a substrate (e.g., paper, plastic sheets, cloth, or the like) or any suitable media.

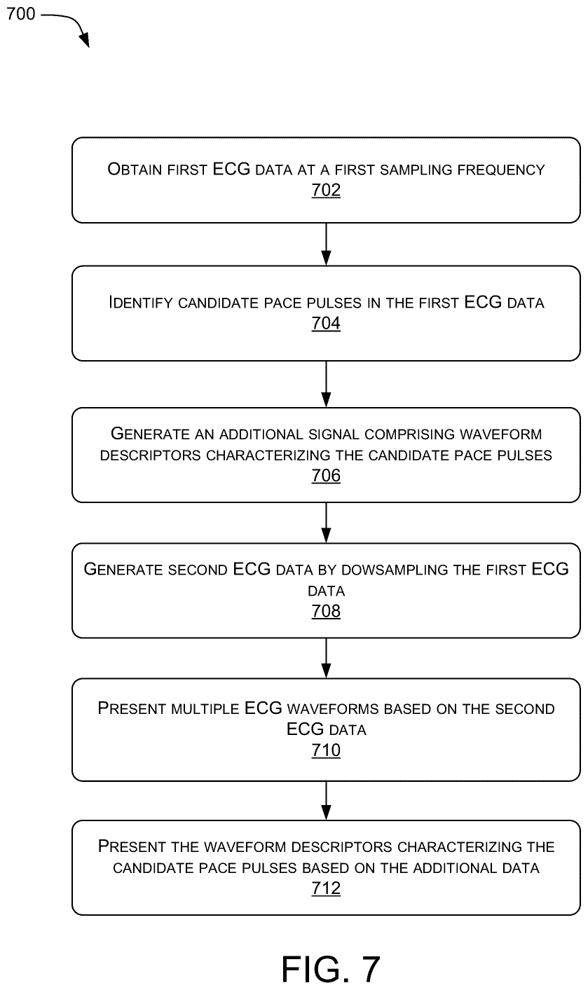

FIG. 7 illustrates a flow chart of an example process 700 for detecting pace pules in the ECG data in accordance with implementations of this disclosure. In some examples, at least some operations of the process 700 may be performed by one or more computing devices, such as an ECG system. Note that the operations of the process 700 are not limited to the orders presented. Rather, the operations of the process 700 can be performed in any proper order. Moreover, at least some operations of the process 700 can be repeated, combined, or split to implement techniques of this disclosure.

At operation 702, the process can include obtaining first ECG data at a first sampling frequency. In some examples, the ECG system can obtain an analog ECG signal via multiple sensors, and convert, via an ADC, the analog ECG signal to the first ECG data at a first sampling frequency. The sensors can be placed in a configuration across a patient's body (such as the chest, the abdomen, shoulders, arms, legs, ankles, wrists, and the like). Additional details are given throughout this disclosure. In some examples, the first ECG data is associated with the first sampling frequency. In some examples, the first sampling frequency can be between 8 kHz and 40 kHz (including the edges of the range). Note that the range of the first sampling frequency is not limited to such a range, but can be any frequency range suitable for detecting the candidate pace pulses.

At operation 704, the process can include identifying, by the ECG system, candidate pace pulses in the first ECG data. In some examples, the ECG system can identify the pace pulses in the first ECG data by identifying a recovery pulse. A time window around the detection time for the candidate pace pulse may be used to bound the capture of additional pace related data. In some examples, the ECG system can identify the pace pulses by detecting edges (such as rising edges, falling edges, and the like) of the pace pulse. In some examples, the ECG system can identify the candidate pace pulses in the first ECG data based on signature pace pulse data. In some examples, the signature pace pulse data can be provided by a third party, such as a pacemaker manufacturer, an ECG database, and the like. In some examples, the candidate pace pulses can include various types of pace pulses such as atrial pace pulses, ventricular pace pules, biventricular pace pules, and the like. Additional details of examples of pace pulses are given throughout this disclosure such as in FIGS. 3A-3F.

At operation 706, the process can include generating, by the ECG system, additional data comprising waveform descriptors characterizing the candidate pace pulses. In some examples, the waveform descriptors characterize the candidate pace pulses in various aspects such as the presence of the candidate pace pulses, the timing of the candidate pace pulses, shapes of the candidate pace pulses, frequency components of the candidate pace pulses, amplitudes of the candidate pace pulses, or the like. Additional details of the waveform descriptors are provided throughout this disclosure.

At operation 708, the process can include generating, by the ECG system, second ECG data by down-sampling the first ECG data. In some instances, the high-fidelity first ECG data may require a lot of resources (such as storage space and bandwidth) to store and/or transmit in real-time or near real-time. However, the ECG system may have limited resources (such as storage space and bandwidth). Therefore, the ECG system can process the first ECG data to reduce the data amount to save resources. The first ECG data can be processed in various ways. In some examples, the ECG system can down-sample the first ECG data at a second sampling frequency to generate the second ECG data. For example, the second sampling frequency is between 500 Hz and 1000 Hz (including the edges of the range). Note that the range of the second sampling frequency is not limited to such a range, but can be any frequency range suitable for processing and storing the first ECG data. In some examples, the second ECG data 130 can include a brief interval of the first ECG data, compressed samples, or a representation such as piecewise splines that can accurately represent the candidate pace pulses.

At operation 710, the process can include presenting multiple ECG waveforms based on the second ECG data. In some examples, the waveform descriptors can characterize shapes of the candidate pace pulses. In some examples, the waveform descriptors can characterize frequency components of the candidate pace pulses. In some examples, the waveform descriptors can characterize amplitudes of the candidate pace pulses. In some examples, the waveform descriptors can include indicators (such as vertical lines at the top and/or bottom of the ECG waveform), indicating the presence and the timing of the candidate pace pulses.

At operation 712, the process can include presenting the one or more waveform descriptors characterizing the candidate pace pulses based on the additional data.

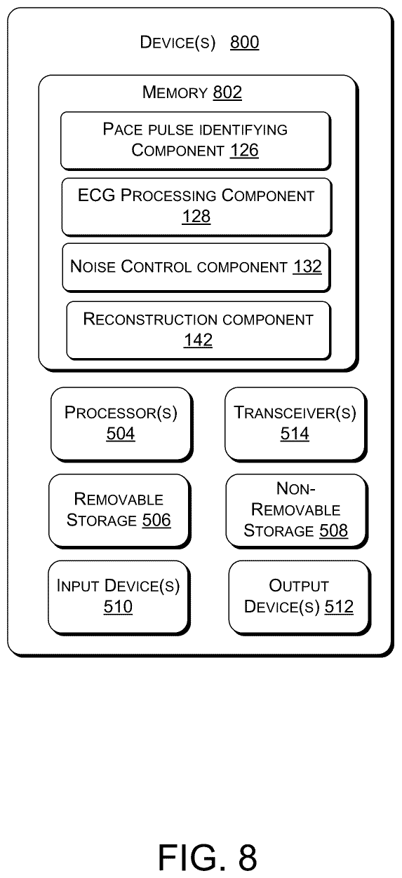

FIG. 8 illustrates at least one example device 800 configured to enable and/or perform the some or all of the functionality discussed herein. Further, the device(s) 800 can be implemented as one or more server computers, a network element on dedicated hardware, as a software instance running on dedicated hardware, or as a virtualized function instantiated on an appropriate platform, such as cloud infrastructure, and the like. It is to be understood in the context of this disclosure that the device(s) 800 can be implemented as a single device or as a plurality of devices with components and data distributed among them.

As illustrated, the device(s) 800 comprises a memory 802. In various embodiments, the memory 802 is volatile (including a component such as Random Access Memory (RAM)), non-volatile (including a component such as Read Only Memory (ROM), flash memory, etc.), or some combination of the two.

The memory 802 may include various components, such as at least one of the pace pulse identifying component 126, the ECG processing component 128, the noise control component 132, or the reconstruction component 142. Any of the pace pulse identifying component 126, the ECG processing component 128, the noise control component 132, or the reconstruction component 142 can include methods, threads, processes, applications, or any other sort of executable instructions. The pace pulse identifying component 126, the ECG processing component 128, the noise control component 132, or the reconstruction component 142 can also include files and databases.

The memory 802 may include various instructions (e.g., instructions in the pace pulse identifying component 126, the ECG processing component 128, the noise control component 132, or the reconstruction component 142), which can be executed by at least one processor 804 to perform operations. In some embodiments, the processor(s) 804 includes a Central Processing Unit (CPU), a Graphics Processing Unit (GPU), or both CPU and GPU, or other processing unit or component known in the art.

The device(s) 800 can also include additional data storage devices (removable and/or non-removable) such as, for example, magnetic disks, optical disks, or tape. Such additional storage is illustrated in FIG. 5 by removable storage 806 and non-removable storage 808. Tangible computer-readable media can include volatile and nonvolatile, removable, and non-removable media implemented in any method or technology for storage of information, such as computer readable instructions, data structures, program modules, or other data. The memory 802, removable storage 806, and non-removable storage 808 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 800. Any such tangible computer-readable media can be part of the device(s) 800.

The device(s) 800 also can include input device(s) 810, such as a keypad, a cursor control, a touch-sensitive display, a voice input device, etc., and output device(s) 810 such as a display, speakers, printers, etc. These devices are well known in the art and need not be discussed at length here. In particular implementations, a user can provide input to the device(s) 800 via a user interface associated with the input device(s) 810 and/or the output device(s) 812.

As illustrated in FIG. 8, the device(s) 800 can also include one or more wired or wireless transceiver(s) 814. For example, the transceiver(s) 814 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, the transceiver(s) 814 can utilize Multiple-Input/Multiple-Output (MIMO) technology. The transceiver(s) 814 can include any sort of wireless transceivers capable of engaging in wireless, Radio Frequency (RF) communication. The transceiver(s) 814 can also include other wireless modems, such as a modem for engaging in Wi-Fi, WiMAX, Bluetooth, or infrared communication.

In some implementations, the transceiver(s) 814 can be used to communicate between various functions, components, modules, or the like, that are comprised in the device(s) 800. For instance, the transceivers 814 may facilitate communications between the vision screening device 104 and other devices storing the pace pulse identifying component 126, the ECG processing component 128, the noise control component 132, or the reconstruction component 142.

The example systems and methods of the present disclosure overcome various deficiencies of known prior art devices. Other embodiments of the present disclosure will be apparent to those skilled in the art from consideration of the specification and practice of the disclosure contained herein. It is intended that the specification and examples be considered as an example only, with a true scope and spirit of the present disclosure being indicated by the following claims.

### Example Clauses

1: A system including: a backend device; and a frontend device including: one or more processors; multiple sensors in communication with the one or more processors and configured to detect an analog electrocardiogram (ECG) signal over time; an analog-to-digital converter (ADC) configured to convert the analog ECG signal to first ECG data, the first ECG data being associated with a first sampling frequency; one or more non-transitory computer-readable media storing computer-executable instructions that, when executed, cause the one or more processors to perform operations including: identifying candidate pace pulses in the first ECG data; generating additional data including waveform descriptors characterizing the candidate pace pulses; generating second ECG data by down-sampling the first ECG data, the second ECG data being associated with a second sampling frequency that is lower than the first sampling frequency; and transmitting the second ECG data and the additional data to the backend device.
2: The system of clause 1, wherein the first sampling frequency is between 8 kHz and 40 kHz, and wherein the second sampling frequency is between 500 Hz and 1000 Hz.
3: The system of either clause 1 or 2, wherein the waveform descriptors characterize shapes of the candidate pace pulses.
4: The system of any one of clauses 1-3, wherein the waveform descriptors characterize frequency components of the candidate pace pulses.
5: The system of any one of clauses 1-4, wherein the waveform descriptors characterize amplitudes of the candidate pace pulses.
6: The system of any one of clauses 1-5, wherein identifying the candidate pace pulses in the first ECG data includes identifying, in the first ECG data, a recovery pulse.
7: The system of any one of clauses 1-6, wherein generating the additional data includes: identifying a portion of the first ECG data including a candidate pace pulse among the candidate pace pulses; and generating a waveform descriptor among the waveform descriptors based on the portion of the first ECG data.
8: The system of clause 7, the operations further including: down-sampling the portion of the first ECG data at a third sampling frequency, the third sampling frequency being higher than the second sampling frequency and lower than the first sampling frequency.
9: The system of any one of clauses 1-8, wherein the waveform descriptor includes the portion of the first ECG data.
10: The system of any one of clauses 1-9, wherein the waveform descriptor includes a representation of the portion of the first ECG data, the representation having a lower fidelity than the portion of the first ECG data.
11: The system of any one of clauses 1-10, wherein the waveform descriptor includes one or more frequency components of the portion of the first ECG data.
12: The system of any one of clauses 1-11, wherein the backend device is configured to determine that a waveform descriptor among the waveform descriptors includes an artifact by: identifying a spike in the waveform descriptor; determining that a paced rhythm is absent within a threshold time period after the spike in the waveform descriptor.
13: The system of clause 12, wherein the backend device is further configured to: modify the additional data by removing the waveform descriptor including the artifact.
14: The system of any one of clauses 1-13, wherein the backend device is further configured to: generate an average pace pulse by performing signal averaging on the candidate pace pules; and compare the average pace pulse to an individual candidate pace pulse.
15: The system of clause 14, wherein the backend device is further configured to: determine a discrepancy between the average pace pulse and the individual candidate pace pulse; determine that the discrepancy is greater than a threshold; and based on determining that the discrepancy is greater than the threshold, modify the additional data by removing the individual candidate pace pulse.
16: The system of any one of clauses 1-15, wherein the backend device is configured to: present multiple ECG waveforms respectively corresponding to multiple leads; and present the waveform descriptors in a user interface element that is separate from the multiple ECG waveforms.
17: The system of any one of clauses 1-16, wherein the backend device is configured to: present multiple ECG waveforms respectively corresponding to multiple leads; and present the waveform descriptors in a user interface element that is visually overlapping the multiple ECG waveforms.
18: The system of clause 16, wherein the backend device includes a display and is configured to: receive an input signal indicating a zoom-in instruction; and based on receiving the input signal, present, on the display, a zoomed-in view of the at least one ECG waveform.
19: The system of any one of clauses 16-18, wherein the backend device includes a display and is configured to: receive an input signal indicating a zoom-in instruction; and based on receiving the input signal, present, on the display, a zoomed-in view of at least one of the waveform descriptors.
20: The system of any one of clauses 16-19, wherein the backend device is configured to: print at least one ECG waveform on a substrate; and print indications of the waveform descriptors on the substrate.
21: A system including one or more processors; one or more non-transitory computer-readable media storing computer-executable instructions that, when executed, cause the one or more processors to perform operations including: obtaining first electrocardiogram (ECG) data associated with a first sampling frequency; identifying candidate pace pulses in the first ECG data; generating additional data including waveform descriptors characterizing the candidate pace pulses; generating second ECG data by down-sampling the first ECG data, the second ECG data being associated with a second sampling frequency that is lower than the first sampling frequency; presenting multiple ECG waveforms based on the second ECG data; and presenting the waveform descriptors characterizing the one or more candidate pace pulses based on the additional data.
22: A frontend device including: one or more processors; one or more non-transitory computer-readable media storing computer-executable instructions that, when executed, cause the one or more processors to perform operations including: obtaining first electrocardiogram (ECG) data associated with a first sampling frequency; identifying candidate pace pulses in the first ECG data; generating additional data including waveform descriptors characterizing the candidate pace pulses; generating second ECG data by down-sampling the first ECG data, the second ECG data being associated with a second sampling frequency that is lower than the first sampling frequency; and transmitting the second ECG data and the additional data to a backend device.
23: A backend device including one or more processors; one or more non-transitory computer-readable media storing computer-executable instructions that, when executed, cause the one or more processors to perform operations including: receiving a second electrocardiogram (ECG) data and additional data including waveform descriptors characterizing candidate pace pulses from a frontend device, the second ECG data being generated based on a first ECG data associated with a first sampling frequency, the second ECG data being associated with a second sampling frequency lower than the first sampling frequency; presenting multiple ECG waveforms based on the second ECG data; and presenting the one or more waveform descriptors characterizing the candidate pace pulses based on the additional data.
24: A method including: obtaining first electrocardiogram (ECG) data associated with a first sampling frequency; identifying candidate pace pulses in the first ECG data; generating additional data including waveform descriptors characterizing the candidate pace pulses; generating second ECG data by down-sampling the first ECG data, the second ECG data being associated with a second sampling frequency that is lower than the first sampling frequency; and transmitting the second ECG data and the additional data to a backend device.
25: The method of clause 24, wherein the first sampling frequency is between 8 kHz and 40 kHz, and wherein the second sampling frequency is between 500 Hz and 1000 Hz.
26: The method of either clause 24 or 25, wherein the waveform descriptors characterize shapes of the candidate pace pulses.
27: The method of any one of clauses 24-26, wherein the waveform descriptors characterize frequency components of the candidate pace pulses.
28: The method of any one of clauses 24-27, wherein the waveform descriptors characterize amplitudes of the candidate pace pulses.
29: The method of any one of clauses 24-28, wherein generating the additional data includes: identifying a portion of the first ECG data including a candidate pace pulse among the candidate pace pulses; and generating a waveform descriptor among the waveform descriptors based on the portion of the first ECG data.
30: The method of clause 29, wherein generating the waveform descriptor among the waveform descriptors includes: down-sampling the portion of the first ECG data at a third sampling frequency, the third sampling frequency being higher than the second sampling frequency and lower than the first sampling frequency.
31: The method of clause 30, wherein the waveform descriptor includes the portion of the first ECG data.
32: The method of any one of clauses 29-31, wherein the waveform descriptor includes a representation of the portion of the first ECG data, the representation having a lower fidelity than the portion of the first ECG data.
33: The method of any one of clauses 29-32, wherein the waveform descriptor includes one or more frequency components of the portion of the first ECG data.
34: The method of any one of clauses 24-33, wherein identifying the candidate pace pulses in the first ECG data includes identifying, in the first ECG data, a recovery pulse.
35: The method of any one of clauses 24-34, wherein generating the additional data includes: identifying portions of the first ECG data including the candidate pace pulses; and generating the waveform descriptors based on the portions of the first ECG data.
36: A method including: receiving a second electrocardiogram (ECG) data and additional data including waveform descriptors characterizing the candidate pace pulses from a frontend device, the second ECG data being generated based on a first ECG data associated with a first sampling frequency, the second ECG data being associated with a second sampling frequency lower than the first sampling frequency; presenting multiple ECG waveforms based on the second ECG data; and presenting the waveform descriptors characterizing the candidate pace pulses based on the additional data.
37: The method of clause 36, further including: determining that a waveform descriptor among the waveform descriptors includes an artifact by: identifying a spike in the waveform descriptor; and determining that a paced rhythm is absent within a threshold time period after the spike in the waveform descriptor.
38: The method of clause 37, further including: modifying the additional data by removing the waveform descriptor including the artifact.
39: The method of any one of clauses 36-38, further including: generating an average pace pulse by performing signal averaging on the candidate pace pulses; and comparing the average pace pulse to an individual pace pulse.
40: The method of clause 39, further including: determining a discrepancy between the average pace pulse and the individual candidate pace pulse; determining that the discrepancy is greater than a threshold; and based on determining that the discrepancy is greater than the threshold, modifying the additional data by removing the individual candidate pace pulse.
41: The method of any one of clauses 36-40, wherein presenting multiple ECG waveforms based on the second ECG data includes presenting the multiple ECG waveforms respectively corresponding to multiple leads; and wherein presenting the waveform descriptors includes presenting the waveform descriptors in a user interface element that is separate from the multiple ECG waveforms.
42: The method of any one of clauses 36-41, wherein presenting multiple ECG waveforms based on the second ECG data includes presenting the multiple ECG waveforms respectively corresponding to multiple leads; and wherein presenting the one or more waveform descriptors includes presenting the waveform descriptors in a user interface element that is visually overlapping the multiple ECG waveforms.
43: The method of any one of clauses 36-42, wherein the backend device includes a display, the method further including: receiving an input signal indicating a zoom-in instruction; and based on receiving the input signal, presenting, on the display, a zoomed-in view of the at least one ECG waveform.
44: The method of any one of clauses 36-43, wherein the backend device includes a display, the method further including: receiving an input signal indicating a zoom-in instruction; and based on receiving the input signal, presenting, on the display, a zoomed-in view of at least one of the waveform descriptors.
45: The method of any one of clauses 36-44, further including: printing at least one ECG waveform on a substrate; and printing indications of the waveform descriptors on the substrate.
46: A method including: obtaining first electrocardiogram (ECG) data associated with a first sampling frequency; identifying candidate pace pulses in the first ECG data; generating additional data including waveform descriptors characterizing the candidate pace pulses; generating second ECG data by down-sampling the first ECG data, the second ECG data being associated with a second sampling frequency that is lower than the first sampling frequency; presenting multiple ECG waveforms based on the second ECG data; and presenting the waveform descriptors characterizing the candidate pace pulses based on the additional data.
47: One or more computer-readable media storing instructions, which when executed by one or more processors, cause the one or more processors to perform operations including: obtaining first electrocardiogram (ECG) data associated with a first sampling frequency; identifying candidate pace pulses in the first ECG data; generating additional data including waveform descriptors characterizing the candidate pace pulses; generating second ECG data by down-sampling the first ECG data, the second ECG data being associated with a second sampling frequency that is lower than the first sampling frequency; and transmitting the second ECG data and the additional data to a backend device.
48: One or more computer-readable media storing instructions, which when executed by one or more processors, cause the one or more processors to perform operations including: receiving a second electrocardiogram (ECG) data and additional data including waveform descriptors characterizing candidate pace pulses from a frontend device, the second ECG data being generated based on a first ECG data associated with a first sampling frequency, the second ECG data being associated with a second sampling frequency lower than the first sampling frequency; presenting multiple ECG waveforms based on the second ECG data; and presenting the one or more waveform descriptors characterizing the candidate pace pulses based on the additional data.
49: One or more computer-readable media storing instructions, which when executed by one or more processors, cause the one or more processors to perform operations including: obtaining first electrocardiogram (ECG) data associated with a first sampling frequency; identifying candidate pace pulses in the first ECG data; generating additional data including waveform descriptors characterizing the candidate pace pulses; generating second ECG data by down-sampling the first ECG data, the second ECG data being associated with a second sampling frequency that is lower than the first sampling frequency; presenting multiple ECG waveforms based on the second ECG data; and presenting the waveform descriptors characterizing the candidate pace pulses based on the additional data.

While the example clauses described above are described with respect to particular implementations, it should be understood that, in the context of this document, the content of the example clauses can be implemented via a method, device, system, a computer-readable medium, and/or another implementation. Additionally, any of examples XX may be implemented alone or in combination with any other one or more of the examples XX.

In some instances, one or more components may be referred to herein as “configured to,” “configurable to,” “operable/operative to,” “adapted/adaptable,” “able to,” and “conformable/conformed to,” etc. Those skilled in the art will recognize that such terms (e.g., “configured to”) can generally encompass active-state components and/or inactive-state components and/or standby-state components unless the context requires otherwise.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

As used herein, the phrase “A is between X and Y” may refer to A being equal to X, A being equal to Y, or A being greater than X and less than Y.

Although the subject matter has been described in language specific to structural features and/or methodological acts, it is to be understood that the subject matter defined in the appended claims is not necessarily limited to the specific features or acts described.

What is claimed is:

1. A system comprising:
a backend device; and
a frontend device comprising:
one or more processors;
multiple sensors in communication with the one or more processors and configured to detect an analog electrocardiogram (ECG) signal over time;
an analog-to-digital converter (ADC) configured to convert the analog ECG signal to first ECG data, the first ECG data being associated with a first sampling frequency;
one or more non-transitory computer-readable media storing computer-executable instructions that, when executed, cause the one or more processors to perform operations comprising:
identifying candidate pace pulses in the first ECG data;
generating additional data comprising waveform descriptors characterizing the candidate pace pulses;
generating second ECG data by down-sampling the first ECG data, the second ECG data being associated with a second sampling frequency that is lower than the first sampling frequency; and
transmitting the second ECG data and the additional data to the backend device.

2. The system of claim 1, wherein the first sampling frequency is between 8 kHz and 40 kHz, and
wherein the second sampling frequency is between 500 Hz and 1000 Hz.

3. The system of claim 1, wherein the waveform descriptors characterize at least one of:
shapes of the candidate pace pulses;
frequency components of the candidate pace pulses; or
amplitudes of the candidate pace pulses.

4. The system of claim 1, wherein identifying the candidate pace pulses in the first ECG data comprises identifying, in the first ECG data, a recovery pulse.

5. The system of claim 1, wherein the waveform descriptor comprises at least one of:
a portion of the first ECG data; or
a representation of the portion of the first ECG data, the representation having a lower fidelity than the portion of the first ECG data.

6. The system of claim 1, wherein the backend device is configured to determine that a waveform descriptor among the waveform descriptors comprises an artifact by:
identifying a spike in the waveform descriptor;
determining that a paced rhythm is absent within a threshold time period after the spike in the waveform descriptor.

7. The system of claim 6, wherein the backend device is further configured to:
modify the additional data by removing the waveform descriptor comprising the artifact.

8. The system of claim 1, wherein the backend device is further configured to:
generate an average pace pulse by performing signal averaging on the candidate pace pulses; and
compare the average pace pulse to an individual candidate pace pulse.

9. The system of claim 8, wherein the backend device is further configured to:
determine a discrepancy between the average pace pulse and the individual candidate pace pulse;
determine that the discrepancy is greater than a threshold; and
based on determining that the discrepancy is greater than the threshold, modify the additional data by removing the individual candidate pace pulse.

10. The system of claim 1, wherein the backend device is configured to:
present multiple ECG waveforms respectively corresponding to multiple leads; and
present the waveform descriptors in a user interface element that is separate from the multiple ECG waveforms or visually overlapping the multiple ECG waveforms.

11. The system of claim 10, wherein the backend device comprises a display and is configured to:
receive an input signal indicating a zoom-in instruction; and
based on receiving the input signal, present, on the display, a zoomed-in view of at least one of the multiple ECG waveforms.

12. A system comprising
one or more processors;
one or more non-transitory computer-readable media storing computer-executable instructions that, when executed, cause the one or more processors to perform operations comprising:
obtaining first electrocardiogram (ECG) data associated with a first sampling frequency;
identifying candidate pace pulses in the first ECG data;
generating additional data comprising waveform descriptors characterizing the candidate pace pulses;
generating second ECG data by down-sampling the first ECG data, the second ECG data being associated with a second sampling frequency that is lower than the first sampling frequency;
presenting multiple ECG waveforms based on the second ECG data; and
presenting the waveform descriptors characterizing the one or more candidate pace pulses based on the additional data.

13. The system of claim 12, wherein the first sampling frequency is between 8 kHz and 40 kHz, and
wherein the second sampling frequency is between 500 Hz and 1000 Hz.

14. The system of claim 12, wherein the waveform descriptors characterize at least one of:
shapes of the candidate pace pulses;
frequency components of the candidate pace pulses; or
amplitudes of the candidate pace pulses.

15. The system of claim 12, wherein generating the additional data comprises:
identifying a portion of the first ECG data comprising a candidate pace pulse among the candidate pace pulses; and
generating a waveform descriptor among the waveform descriptors based on the portion of the first ECG data, the waveform descriptor comprising a representation of the portion of the first ECG data, the representation having a lower fidelity than the portion of the first ECG data.

16. A method comprising:
obtaining first electrocardiogram (ECG) data associated with a first sampling frequency;
identifying candidate pace pulses in the first ECG data;
generating additional data comprising waveform descriptors characterizing the candidate pace pulses;
generating second ECG data by down-sampling the first ECG data, the second ECG data being associated with a second sampling frequency that is lower than the first sampling frequency; and
transmitting the second ECG data and the additional data to a backend device.

17. The method of claim 16, wherein the first sampling frequency is between 8 kHz and 40 kHz, and
wherein the second sampling frequency is between 500 Hz and 1000 Hz.

18. The method of claim 16, wherein the waveform descriptors characterize at least one of:
shapes of the candidate pace pulses;
frequency components of the candidate pace pulses; or
amplitudes of the candidate pace pulses.

19. The method of claim 16, wherein generating the additional data comprises:
identifying a portion of the first ECG data comprising a candidate pace pulse among the candidate pace pulses; and
generating a waveform descriptor among the waveform descriptors based on the portion of the first ECG data, the waveform descriptor comprising a representation of the portion of the first ECG data, the representation having a lower fidelity than the portion of the first ECG data.

20. The method of claim 16, further comprising:
generating an average pace pulse by performing signal averaging on the candidate pace pulses;
determining a discrepancy between the average pace pulse and individual candidate pace pulses;
determining that the discrepancy is greater than a threshold; and
based on determining that the discrepancy is greater than the threshold, modifying the additional data by removing the individual candidate pace pulse.
